(19) United States
(12) Patent Application Publication
(10) Pub. No.: US 2024/0425868 A1
(43) Pub. Date: Dec. 26, 2024

(54) ENGINEERED PLASMODIA AND RELATED METHODS

(71) Applicant: Flagship Pioneering Innovations VII, LLC, Cambridge, MA (US)

(72) Inventors: Ellen Lovisa Larsdotter AFZELIUS, Belmont, MA (US); Hozefa Saifuddin BANDUKWALA, Southborough, MA (US); Brendan Peltonen LEHNERT, Cambridge, MA (US); Erika GONZALEZ RODRIGUEZ, Somerville, MA (US)

(21) Appl. No.: 18/752,946

(22) Filed: Jun. 25, 2024

Related U.S. Application Data
(60) Provisional application No. 63/510,259, filed on Jun. 26, 2023.
(60) Provisional application No. 63/557,731, filed on Feb. 26, 2024.
(60) Provisional application No. 63/641,832, filed on May 2, 2024.

(51) Int. Cl. C12N 15/79; C07K 14/705; C07K 16/24

(52) U.S. Cl. CPC C12N 15/79; C07K 14/70578; C07K 16/241; C07K 2317/14; C07K 2319/30

(57) ABSTRACT

Provided herein are engineered Plasmodia comprising a heterologous nucleic acid molecule encoding a therapeutic protein and compositions (e.g., pharmaceutical compositions) comprising the same; as well as methods of making engineered Plasmodia comprising a heterologous nucleic acid molecule encoding a therapeutic protein and compositions (e.g., pharmaceutical compositions) comprising the same. The engineered Plasmodia provided herein are useful e.g., in pharmaceutical compositions and methods of treating diseases.

### RELATED APPLICATIONS

This application claims priority to each of U.S. Ser. No. 63/510,259, filed Jun. 26, 2024, U.S. Ser. No. 63/557,731, filed Feb. 26, 2024; and U.S. Ser. No. 63/641,832, filed May 2, 2024; the entire contents of each of which is incorporated herein by reference.

### SEQUENCE LISTING

The instant application contains a Sequence Listing which has been submitted electronically in XML format and is hereby incorporated by reference in its entirety. Said XML copy, created on Jun. 25, 2024, is named 62801.21US01 Sequence Listing.xml and is 6,493 bytes in size.

### 1. FIELD

This disclosure relates to engineered Plasmodia (e.g., genetically engineered Plasmodia) comprising a heterologous nucleic acid molecule encoding a therapeutic protein. The disclosure further relates to methods of making and utilizing the same.

### 2. BACKGROUND

Plasmodia are single cell obligate intracellular parasites; and are known to infect vertebrates, including humans, as well as insects (e.g., mosquitos). Certain pathogenic species of Plasmodia are the causative agent of malaria in humans and other animals (e.g., Plasmodium falciparum). All Plasmodia have a similar vector-host life cycle, wherein an infected vector (e.g., a mosquito) transmits the parasite to a host (e.g., a human). Inside the host, the parasites infect the liver (the liver stage) and subsequently the red blood cells (RBCs) (the blood stage). A portion of the parasites differentiate into gametocytes, which are ingested by a new vector (e.g., a mosquito) and are ultimately transmitted to a new host (e.g., a human), completing the parasite life cycle. In the host (e.g., human) those parasites that do not differentiate into gametocytes grow and rupture infected RBCs resulting in the continued infection of new RBCs in circulation. The blood stage of the Plasmodia life cycle is responsible for the clinical manifestations of malaria.

### 3. SUMMARY

Provided herein are, inter alia, engineered Plasmodia comprising a heterologous nucleic acid molecule encoding a therapeutic protein; methods of manufacturing; pharmaceutical compositions; and methods of use including, e.g., methods of making a therapeutic protein in vitro and methods of treating a disease utilizing the therapeutic protein.

As such, in one aspect, provided herein are engineered Plasmodium comprising a heterologous nucleic acid molecule within a genome of the Plasmodium, wherein the heterologous nucleic acid molecule encodes a therapeutic protein.

In some embodiments, the engineered Plasmodium is non-pathogenic in humans. In some embodiments, the engineered Plasmodium is auxotrophic. In some embodiments, the engineered Plasmodium does not contain a functional apicoplast.

In some embodiments, the engineered Plasmodium is incapable of synthesizing one or more isoprenoid precursor. In some embodiments, the engineered Plasmodium is incapable of synthesizing isopentenyl pyrophosphate (IPP). In some embodiments, the genome of the engineered Plasmodium comprises a functional deletion of one or more genes that encodes an apicoplast protein.

In some embodiments, the genome of the Plasmodium comprises a functional deletion of one or more genes that encodes a virulence protein. In some embodiments, the engineered Plasmodium is a Plasmodium falciparum (P. falciparum), and wherein the genome of the P. falciparum comprises a functional deletion of the Plasmodium falciparum erythrocyte membrane protein 1 (PfEMP1) gene.

In some embodiments, the therapeutic protein is a secreted protein. In some embodiments, the therapeutic protein is an intracellular protein. In some embodiments, the therapeutic protein is a transmembrane protein. In some embodiments, the transmembrane therapeutic protein specifically binds a transmembrane protein expressed on a target cell. In some embodiments, the therapeutic protein is an antibody, an enzyme, a cytokine, a chemokine, a hormone, a growth factor, a fusion protein, or an immunogen, or a functional fragment and/or functional variant of any of the foregoing. In some embodiments, the therapeutic protein is an intracellular enzyme.

In some embodiments, the Plasmodium is a P. falciparum, a Plasmodium malariae (P. malariae), a Plasmodium vivax (P. vivax), a Plasmodium ovale (P. ovale), a Plasmodium knowlesi (P. knowlesi), or a Plasmodium yoelii (P. yoelii).

In some embodiments, the heterologous nucleic acid molecule is positioned within the nuclear genome, the apicoplast genome, or the mitochondrial genome of the Plasmodium.

In some embodiments, the heterologous nucleic acid molecule is integrated within any one or more of the genes set forth in Table 2.

In some embodiments, the heterologous nucleic acid molecule is integrated within any one or more of the following genes: GAPDH, HSP70, MESA, MSP9, HGPRT, ENO, HSP90, PMT, H2B, LDH, FBPA, GBP130, ACT1, CK1, PyrK, NT1, EBA175, ETRAMP2, ALBA1, BIP, nPrx, RPL2, MSP1, RESA, RESA3, ETRAMP11.2, eIF4A, ETRAMP5, H2B.Z, PREBP, RPP1, PDI8, EXP1, RhopH3, RAN, P23, PNP, NAPS, IMC1g, RPS19, NAPL, RPL5, RPL32, EXP2, LCN, EF-1beta, CYP19A, H2A, H2A.Z, AdoMetDC/ODC, RPL38, TRX1, eEF2, RPL3, PABP1, RPL21, TCTP, PfP0, H3, 14-3-3I, RPS19, EIF3A, MyoA, RPL29, SERA5, PK5, HSP110c, RPL27, ADA, PfpUB, SRSF4, PKAr, CEPT, VAR2CSA, RACK1, IMC1c, HMGB2, RPS27, REX1, Pfs16, HAD1, RPS4, RPS2, ADF1, ApiAP2, RPS24, GBP2, ETRAMP10, RPL39, RPS3A, SAMS, PV1, ETRAMP4, PGM1, ERC, RPS11, RPS9, RPL23, RPS26, RPS12, HSP70x, SBP1, RPL4, PGK, MIF, ETRAMP14, or SAHH.

In some embodiments, the engineered Plasmodium is a P. falciparum, and the heterologous nucleic acid molecule is positioned within the Pf47 gene or the HAP110 gene. In some embodiments, the heterologous nucleic acid molecule further encodes a signal peptide.

In some embodiments, at least two copies of the heterologous nucleic acid molecule are integrated into a single gene (e.g., a gene set forth in Table 2); and/or at least one copy of the heterologous nucleic acid molecule is integrated into at least two different genes (e.g., two different genes set forth in Table 2).

In one aspect, provided herein are methods of making an engineered Plasmodium, the method comprising: (a) culturing a reference Plasmodium; (b) genetically modifying the reference Plasmodium to introduce a heterologous nucleic acid molecule encoding a therapeutic protein into a genome of the reference Plasmodium; to thereby make an engineered Plasmodium.

In some embodiments, step (a) comprises culturing a reference Plasmodium in a composition comprising hRBCs. In some embodiments, prior to step (b), the reference Plasmodium is isolated from the composition comprising the hRBCs.

In some embodiments, the method further comprises isolating the engineered Plasmodium.

In some embodiments, step (b) comprises introducing into the reference Plasmodium a genetic engineering system. In some embodiments, the genetic engineering system is a Clustered Regularly Interspaced Short Palindromic Repeats (CRISPR)/Cas system, a zinc-finger nuclease (ZFN) system, a transcription activator-like effector nuclease (TALEN), or a meganuclease system.

In some embodiments, the composition comprising hRBCs is whole human blood or a composition comprising isolated hRBCs.

In some embodiments, the engineered Plasmodium is non-pathogenic in humans. In some embodiments, the engineered Plasmodium is auxotrophic. In some embodiments, the engineered Plasmodium does not contain a functional apicoplast. In some embodiments, the engineered Plasmodium is incapable of synthesizing one or more isoprenoid precursor. In some embodiments, the engineered Plasmodium is incapable of synthesizing IPP.

In some embodiments, the genome of the engineered Plasmodium comprises a functional deletion of one or more genes that encodes an apicoplast protein. In some embodiments, the genome of the Plasmodium comprises a functional deletion of one or more genes that encodes a virulence protein. In some embodiments, the engineered Plasmodium is a P. falciparum, and wherein the genome of the P. falciparum comprises a functional deletion of the Plasmodium falciparum erythrocyte membrane protein 1 (PfEMP1) gene.

In some embodiments, the therapeutic protein is a secreted protein. In some embodiments, the therapeutic protein is an intracellular protein. In some embodiments, the therapeutic protein is a transmembrane protein. In some embodiments, the therapeutic protein specifically binds a membrane protein expressed on a target cell. In some embodiments, the therapeutic protein is an antibody, an enzyme, a cytokine, a chemokine, a hormone, a growth factor, a fusion protein, or an immunogen, or a functional fragment or functional variant of any of the foregoing. In some embodiments, the therapeutic protein is an intracellular enzyme.

In some embodiments, the Plasmodium is a P. falciparum, a P. malariae, a P. vivax, a P. ovale, a P. knowlesi, or a P. yoelii.

In some embodiments, the heterologous nucleic acid molecule is integrated within any one or more of the genes set forth in Table 2.

In some embodiments, the heterologous nucleic acid molecule is integrated within any one or more of the following genes: GAPDH, HSP70, MESA, MSP9, HGPRT, ENO, HSP90, PMT, H2B, LDH, FBPA, GBP130, ACT1, CK1, PyrK, NT1, EBA175, ETRAMP2, ALBA1, BIP, nPrx, RPL2, MSP1, RESA, RESA3, ETRAMP11.2, eIF4A, ETRAMP5, H2B.Z, PREBP, RPP1, PDI8, EXP1, RhopH3, RAN, P23, PNP, NAPS, IMC1g, RPS19, NAPL, RPL5, RPL32, EXP2, LCN, EF-1beta, CYP19A, H2A, H2A.Z, AdoMetDC/ODC, RPL38, TRX1, eEF2, RPL3, PABP1, RPL21, TCTP, PfP0, H3, 14-3-3I, RPS19, EIF3A, MyoA, RPL29, SERA5, PK5, HSP110c, RPL27, ADA, PfpUB, SRSF4, PKAr, CEPT, VAR2CSA, RACK1, IMC1c, HMGB2, RPS27, REX1, Pfs16, HAD1, RPS4, RPS2, ADF1, ApiAP2, RPS24, GBP2, ETRAMP10, RPL39, RPS3A, SAMS, PV1, ETRAMP4, PGM1, ERC, RPS11, RPS9, RPL23, RPS26, RPS12, HSP70x, SBP1, RPL4, PGK, MIF, ETRAMP14, or SAHH.

In some embodiments, the heterologous nucleic acid molecule is positioned within the nuclear genome, the apicoplast genome, or the mitochondrial genome of the Plasmodium. In some embodiments, the engineered Plasmodium is a P. falciparum and the heterologous nucleic acid molecule encoding a therapeutic protein is introduced within the Pf47 gene or the HAP110 gene of the reference Plasmodium.

In some embodiments, at least two copies of the heterologous nucleic acid molecule are integrated into a single gene (e.g., a gene set forth in Table 2); and/or at least one copy of the heterologous nucleic acid molecule is integrated into at least two different genes (e.g., two different genes set forth in Table 2).

In some embodiments, the heterologous nucleic acid molecule further encodes a signal peptide.

In one aspect, provided herein are methods of making a therapeutic protein, the method comprising: culturing an engineered Plasmodium described herein in a composition comprising hRBCs, under conditions and for a period of time sufficient for (i) infection of the hRBCs by the engineered Plasmodium and (ii) expression the therapeutic protein, to thereby make a therapeutic protein.

In some embodiments, the method further comprises isolating the therapeutic protein. In some embodiments, the method further comprises purifying the therapeutic protein.

In some embodiments, the therapeutic protein is secreted from the hRBCs. In some embodiments, the therapeutic protein is not secreted from the hRBCs. In some embodiments, the culturing results in the rupture of hRBCs infected with the engineered Plasmodium. In some embodiments, the rupture results in the release of the therapeutic protein.

In some embodiments, the composition comprising hRBCs is whole human blood or a composition comprising isolated hRBCs.

In some embodiments, the composition comprising hRBCs is whole human blood. In some embodiments, the method further comprises isolating the serum of the whole human blood. In some embodiments, the serum comprises the therapeutic protein.

In some embodiments, the method further comprises measuring the concentration of therapeutic protein produced.

In one aspect, provided herein are methods of making a therapeutic protein, the method comprising culturing hRBCs comprising an engineered Plasmodium described herein in the presence of a population of hRBCs under conditions and for a period of time sufficient for expression of the therapeutic protein, to thereby make a therapeutic protein.

In some embodiments, the hRBCs comprising the engineered Plasmodium are cultured in the presence of the population of RBCs under conditions and for a period of time sufficient for (i) release of the engineered Plasmodium from the hRBCs, (ii) infection of the population of hRBCs by the engineered Plasmodium, and (iii) expression of the therapeutic protein.

In some embodiments, the method further comprises isolating the therapeutic protein. In some embodiments, the method further comprises purifying the therapeutic protein.

In some embodiments, the therapeutic protein is secreted from the hRBCs. In some embodiments, the therapeutic protein is not secreted from the hRBCs. In some embodiments, the culturing results in the rupture of hRBCs infected with the engineered Plasmodium. In some embodiments, the rupture results in the release of the therapeutic protein.

In some embodiments, the composition comprising hRBCs is whole human blood or a composition comprising isolated hRBCs. In some embodiments, the composition comprising hRBCs is whole human blood. In some embodiments, the method further comprises isolating the serum of the whole human blood. In some embodiments, the serum comprises the therapeutic protein.

In some embodiments, the method further comprises measuring the concentration of therapeutic protein produced.

In one aspect, provided herein are pharmaceutical compositions comprising an engineered Plasmodium described herein or a therapeutic protein made by a method described herein, and a pharmaceutically acceptable excipient. In some embodiments, the pharmaceutically acceptable excipient is serum (e.g., a from a subject in need of administration of the therapeutic protein).

In one aspect, provided herein are kits comprising a vessel (e.g., a syringe) (i) configured for drawing blood from a human patient and (ii) comprising an engineered Plasmodium comprising in its genome a heterologous nucleic acid molecule encoding a therapeutic protein (e.g., an engineered Plasmodium described herein).

In some embodiments, the kit further comprises any one or more (e.g., 1, 2, or 3) of (i) a temperature control device configured to warm the vessel (e.g., optionally comprising a battery); (ii) a device for measuring protein concentration (e.g., a spectrophotometer capable of measuring optical absorbance at 280 nm); and/or (iii) an injection or infusion device (e.g., a syringe) configured to introduce a solution comprising a therapeutic protein into a subject, wherein the injection or infusion device is compatible with the vessel such that the device can be attached to the vessel for administration of the contents of the vessel into the subject.

In some embodiments, the vessel holds from about 1-50 mL (e.g., 1-40 mL, 1-30 mL, 1-20 mL, 1-10 mL, 1-5 mL, 5-50 mL, 5-40 mL, 5-30 mL, 5-20 mL, 5-10 mL) of blood. In some embodiments, the vessel comprises a population of hRBCs comprising the engineered Plasmodium. In some embodiments, the vessel is capable of supporting the culture of cells (e.g., RBCs) (e.g., RBCs obtained from a subject)).

In one aspect, provided herein are kits comprising an engineered Plasmodium described herein, a population of hRBCs comprising an engineered Plasmodium described herein, a therapeutic protein made by a method described herein, or a pharmaceutical composition described herein.

In some embodiments, the kit comprises a population of hRBCs comprising an engineered Plasmodium described herein.

In some embodiments, the kit comprises a vessel (e.g., a syringe) comprising the population of hRBCs. In some embodiments, the vessel (e.g., a syringe) is compatible with a device capable of obtaining a blood sample from a subject (e.g., a butterfly needle, syringe, etc.). In some embodiments, the vessel is capable of supporting the culture of cells (e.g., hRBCs) (e.g., obtained from a subject).

In some embodiments, the kit comprises a temperature control device (e.g., warmer). In some embodiments, the temperature control device (e.g., warmer) is compatible with the vessel, such that when the temperature control device (e.g., warmer) is attached or otherwise associated with the vessel, the temperature control device (e.g., warmer) can control the temperature of the vessel (e.g., modulate (e.g., warm) the temperature of the contents of the vessel).

In some embodiments, the kit comprises a device capable of measuring the concentration of a protein.

In some embodiments, the kit comprises an administration device capable of administering a therapeutic protein into a subject. In some embodiments, the administration device is compatible with the vessel (e.g., a syringe) such that the device can be attached to the vessel (e.g., a syringe) for administration of the contents of the vessel (e.g., a syringe) to a subject.

In one aspect, provided herein are methods of delivering a therapeutic protein to a subject, the method comprising: administering to the subject a therapeutic protein described herein or a pharmaceutical composition described herein, to thereby deliver the therapeutic protein to the subject.

In one aspect, provided herein are methods of delivering an engineered Plasmodium a subject, the method comprising: administering to the subject an engineered Plasmodium described herein or a pharmaceutical composition described herein, to thereby deliver the engineered Plasmodium to the subject.

In one aspect, provided herein are methods of treating, ameliorating, or preventing a disease in a subject in need thereof, the method comprising: administering to the subject an engineered Plasmodium described herein or a pharmaceutical composition described herein, to thereby treat, ameliorate, or prevent the disease in the subject.

In some embodiments, the method comprises administering to the subject the engineered Plasmodium described herein, wherein the subject does not exhibit or only exhibits a mild symptomatic immune response to the engineered Plasmodium.

In some embodiments, the method comprises administering to the subject the engineered Plasmodium described herein and co-administering a cognate compound of the auxotrophic engineered Plasmodium to the subject.

In some embodiments, if the subject exhibits an immune response or more than a mild immune response to the auxotrophic engineered Plasmodium withholding or discontinuing co-administration of the cognate compound.

In one aspect, provided herein are methods of treating, ameliorating, or preventing a disease in a subject in need thereof, the method comprising: administering to the subject a therapeutic protein described herein or a pharmaceutical composition described herein, to thereby treat, ameliorate, or prevent the disease in the subject.

In some embodiments, the method comprises administering to the subject the therapeutic protein made by a method described herein, wherein the hRBCs utilized in a method described herein, are autologous or allogenic to the subject.

In one aspect, provided herein are methods of treating, ameliorating, or preventing a disease in a subject in need thereof, the method comprising (a) obtaining a composition of hRBCs comprising an engineered Plasmodium described herein; (b) obtaining a sample of whole blood from the subject; (c) culturing the composition in the presence of the sample under conditions and for a period of time sufficient to allow for expression of the therapeutic protein; (d) isolating the serum from the hRBCs of the culture, wherein the serum comprises the expressed therapeutic protein; and (e) administering either (i) at least a portion of the serum comprising the therapeutic protein or (ii) the therapeutic protein, to the subject, to thereby treat, ameliorate, or prevent the subject.

In some embodiments, the composition and the same are cultured under conditions and for a period of time sufficient for (i) release of the engineered Plasmodium from the hRBCs of the composition, (ii) infection of the population of hRBCs in the sample by the engineered Plasmodium, and (iii) expression of the therapeutic protein.

In some embodiments, the method further comprises isolating the therapeutic protein prior to administration to the subject.

In some embodiments, the method further comprises purifying the therapeutic protein prior to administration to the subject.

In some embodiments, the therapeutic protein is secreted from the hRBCs. In some embodiments, the therapeutic protein is not secreted from the hRBCs. In some embodiments, the culturing results in the rupture of hRBCs infected with the engineered Plasmodium. In some embodiments, the rupture results in the release of the therapeutic protein.

In some embodiments, the method further comprises measuring the concentration of therapeutic protein produced prior to administration to the subject.

In some embodiments, the method further comprises measuring the concentration of therapeutic protein to determine the appropriate volume for administration to the subject.

In one aspect, provided herein are the engineered Plasmodium described herein, the therapeutic protein made by a method described herein, or a pharmaceutical composition described herein for use in a method of treating, ameliorating, or preventing a disease in a subject in need thereof, the method comprising administering to the subject an engineered Plasmodium described herein, a therapeutic protein made by a method described herein, or a pharmaceutical composition described herein. In some embodiments, the subject does not exhibit or only exhibits a mild symptomatic immune response to the engineered Plasmodium. In some embodiments, the hRBCs utilized in a method described herein, are autologous or allogenic to the subject.

In one aspect, provided herein are the engineered Plasmodium described herein for use in a method of treating, ameliorating, or preventing a disease in a subject in need thereof, the method comprising (a) obtaining a composition of hRBCs comprising an engineered Plasmodium described herein; (b) obtaining a sample of whole blood from the subject; (c) culturing the composition in the presence of the sample under conditions and for a period of time sufficient to allow for expression of the therapeutic protein, (d) isolating the serum from the hRBCs of the culture, wherein the serum comprises the expressed therapeutic protein; and I administering either (i) at least a portion of the serum comprising the therapeutic protein or (ii) the therapeutic protein, to the subject.

In one aspect, provided herein is the use of an engineered Plasmodium described herein, a therapeutic protein made by a method described herein, or a pharmaceutical composition described herein for the manufacture of a medicament.

In one aspect, provided herein is the use of an engineered Plasmodium described herein, a therapeutic protein made by a method described herein, or a pharmaceutical composition described herein for the manufacture of a medicament for the treatment, amelioration, or prevention a disease in a subject in need thereof. In some embodiments, the subject does not exhibit or only exhibits a mild symptomatic immune response to the engineered Plasmodium. In some embodiments, the hRBCs are autologous or allogenic to the subject.

In one aspect, provided herein is the use of an engineered Plasmodium described herein for the manufacture of a medicament for use in a method a method of treating, ameliorating, or preventing a disease in a subject in need thereof, the method comprising (a) obtaining a composition of hRBCs comprising an engineered Plasmodium described herein; (b) obtaining a sample of whole blood from the subject; (c) culturing the composition in the presence of the sample under conditions and for a period of time sufficient to allow for expression of the therapeutic protein, (d) isolating the serum from the hRBCs of the culture, wherein the serum comprises the expressed therapeutic protein; and (e) administering either (i) at least a portion of the serum comprising the therapeutic protein or (ii) the therapeutic protein, to the subject.

### 4. BRIEF DESCRIPTION OF THE DRAWINGS

FIG. 1A is a graphical representation of a kit described herein comprising a genetically engineered Plasmodium comprising in its genome a heterologous nucleic acid molecule encoding a therapeutic protein (e.g., wherein the genetically engineered Plasmodium is within red blood cells (RBCs) in the vessel (e.g., pelleted within the vessel)). The figure further shows exemplary additional optional kit components including (i) a temperature control device (e.g., configured to warm the vessel (e.g., optionally comprising a battery)); (ii) a device for measuring protein concentration (e.g., a spectrophotometer (e.g., capable of measuring optical absorbance at 280 nm)); and (iii) an administration device (e.g., an injection or infusion device (e.g., a syringe)) configured to introduce a solution comprising a therapeutic protein into a subject (e.g., an administration device (e.g., an injection device) compatible with the vessel such that the device can be attached to the vessel for administration of the contents of the vessel into a subject).

FIG. 1B is a graphical representation of an exemplary process of manufacturing and administering a therapeutic protein to a subject utilizing an engineered Plasmodium described herein. First, step (i) depicts the obtaining and shipping (e.g., at 4° C.) of a vessel comprising a genetically engineered Plasmodium comprising in its genome a heterologous nucleic acid molecule encoding a therapeutic protein (e.g., wherein the genetically engineered Plasmodium is within red blood cells (RBCs) in the vessel (e.g., pelleted within the vessel)) (e.g., as depicted in FIG. 1A). Step (ii) depicts the drawing of blood (e.g., approximately 10 mL) from the subject in need of administration of (e.g., treatment with) the therapeutic protein into the vessel (from step (i)). Step (iii) depicts the mixing of the subject's blood with the genetically engineered Plasmodium (e.g., RBCs containing the engineered Plasmodium) in the vessel and incubation (e.g., at approximately 37° C. for 48 hours) (e.g., utilizing a temperature control device provided in a kit described herein (e.g., as described in FIG. 1A)). Step (iv) shows the manual insertion of a valve into the vessel to separate the serum from the RBCs within the vessel. In such embodiments, centrifugation of the vessel would not be required to separate the serum from the RBCs in the vessel. Step (v) depicts the measurement of the concentration of the therapeutic protein within the serum in the vessel (e.g., utilizing a device that can measure protein concentration (e.g., a spectrophotometer capable of measuring optical absorbance at 280 nm) e.g., provided in a kit described herein (e.g., as described in FIG. 1A)). The amount of the solution within the vessel (comprising the therapeutic protein) needed to be delivered to the subject can be determined and adjusted based on the determined concentration of the of the therapeutic protein in the serum. Step (vi) depicts the administration of the adjusted dose of the therapeutic protein to the subject (e.g., via injection (e.g., utilizing an administration device (e.g., a syringe) provided in a kit described herein (e.g., as described in FIG. 1A)).

FIG. 2 is a photograph of an electrophoresis gel showing the PCR products derived from Plasmodia genetically engineered to express an PEXEL-TNFR2 ECD-Fc fusion protein or an anti-TNFα antibody. The image shows bands corresponding to the anticipated sizes of the integrated constructs—a distinct band at approximately 2.8 Kb for the PEXEL-TNFR2 ECD-Fc fusion protein (indicated with an arrow) and another distinct band at around 3.4 Kb for the anti-TNFα antibody (indicated with an arrow), thereby confirming accurate genomic incorporation of both constructs into the genome of the Plasmodia.

FIG. 3 is a photograph of a western blot of proteins derived from the supernatants of RBC cultured with Plasmodia genetically engineered to express an PEXEL-TNFR2 ECD-Fc fusion protein or an anti-TNFα antibody. The image shows the presence of a protein band at approximately 150 kDa (for the anti-TNFα antibody) and approximately 148 KDa (for the PEXEL-TNFR2 ECD-Fc fusion protein) confirming the positive protein expression of the anti-TNFα antibody and the TNFR2 ECD-Fc fusion protein in the supernatant of transgenic plasmodium parasites encoding each protein. A commercially available reagent version of the anti-TNFα antibody was used as positive control and supernatant from non-transfected P. falciparum 3D7 parasites was used as negative control.

FIG. 4 is a bar graph showing the TNF binding capacity (measured in ng/ml), for each of the indicated clones (C1, C2, C3, C4, C5, C6, C7, C8, and C9) transfected with a construct encoding the anti-TNFα antibody. The measurement serves as an indicator of the functional anti-TNFα antibody protein expression in each clone. The parental control and polyclonal population served as baseline references.

FIG. 5 is a bar graph showing the TNF binding capacity (measured in ng/ml), for each of the indicated clones (C1, C2, C3, and C4) transfected with a construct encoding the TNFR2 ECD-Fc fusion protein. The measurement serves as an indicator of the functional TNFR2 ECD-Fc fusion protein expression in each clone. The parental control and polyclonal population served as baseline references.

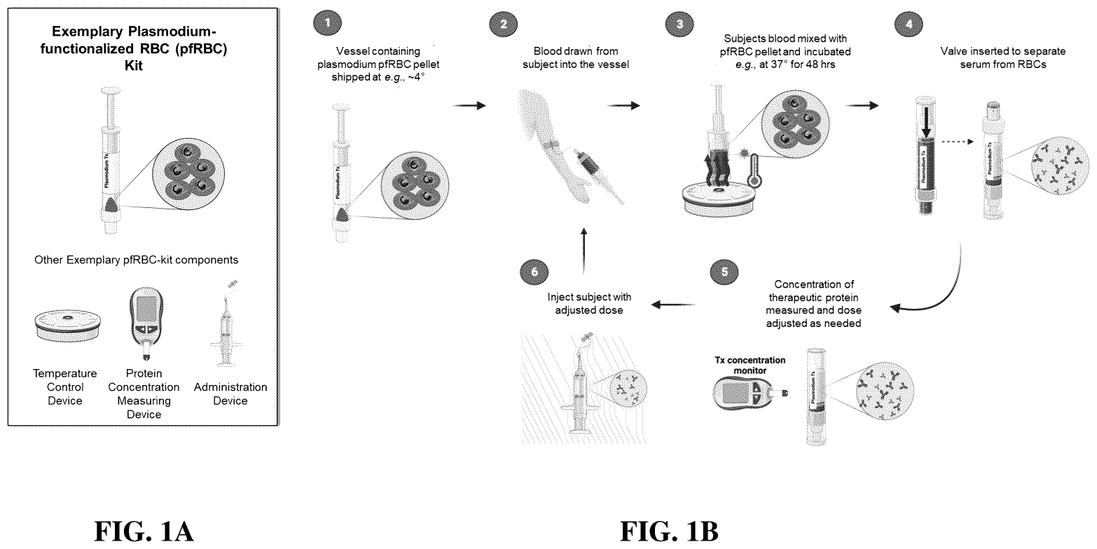

### 5. DETAILED DESCRIPTION

The inventors have, inter alia, identified engineered Plasmodia (e.g., non-pathogenic) as a vehicle for the manufacture and/or delivery (e.g., sustained delivery) of therapeutic proteins, e.g., in vitro, ex vivo, or in vivo. Accordingly, the engineered Plasmodia (e.g., genetically engineered Plasmodia) described herein are useful for the production of proteins, e.g., therapeutic proteins, for delivery of proteins to a human, e.g., for the treatment of diseases (e.g., diseases that can be treated through the administration of a therapeutic protein). As such, the current disclosure provides, inter alia, engineered Plasmodia (e.g., genetically engineered) comprising a heterologous nucleic acid molecule encoding a protein, e.g., a therapeutic protein, and pharmaceutical compositions comprising the same for the use in delivering proteins to a subject, e.g., for treating diseases.

### Table of Contents

5.1 Definitions
5.2 Engineered Plasmodia
5.2.1 Plasmodia Species
5.2.2 Pathogenic & Non-Pathogenic Plasmodia & Methods of Making
5.2.2.1 Auxotrophic Plasmodia
5.2.2.2 Attenuated Plasmodia
5.2.3 Heterologous Nucleic Acid Molecules
5.2.4 Therapeutic Proteins & Polypeptides
5.3 Methods of Making Engineered Plasmodia
5.3.1 Genetic Engineering Systems
5.3.1.1 Engineered CRISPR/Cas Systems
5.3.1.2 Engineered TALENs
5.3.1.3 Engineered ZFNs
5.3.1.4 Engineered Meganucleases
5.4 Scalable Manufacturing of Engineered Plasmodia
5.5 Methods of Making Therapeutic Proteins In Vitro Using Plasmodia
5.6 Pharmaceutical Compositions
5.7 Methods of Use
5.7.1 Delivery
5.7.2 Treatment, Amelioration, and Prevention of Diseases
5.8 Kits

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” should be assumed to be within an acceptable error range for that particular value or composition.

Where proteins and/or polypeptides are described herein, it is understood that nucleic acid molecules (e.g., RNA (e.g., mRNA) or DNA nucleic acid molecules) encoding the protein are also provided herein.

Where proteins, polypeptides, nucleic acid molecules, vectors, carriers, etc. are described herein, it is understood that isolated forms of the proteins, polypeptides, nucleic acid molecules, vectors, carriers, etc. are also provided herein.

Where proteins, polypeptides, nucleic acid molecules, etc. are described herein, it is understood that recombinant forms of the proteins, polypeptides, nucleic acid molecules, etc. are also provided herein.

Where proteins or sets of proteins are described herein, it is understood that both proteins comprising the primary structure are provided herein as well as proteins folded into their three-dimensional structure (i.e., tertiary or quaternary structure) are provided herein.

Where proteins are described herein, it is understood that functional variants, functional fragment, and functional variants and fragments are provided herein. It is understood that the terms “functional fragment or variant,” “functional fragment or functional variant,” “functional fragment and/or functional variant” and the like—provide specific disclosure of proteins that are functional fragments, functional variants, and functional fragments and functional variants (of the reference protein).

As used herein, the term “administering” refers to the physical introduction of an agent, e.g., a therapeutic agent (or a precursor of the therapeutic agent that is metabolized or altered within the body of the subject to produce the therapeutic agent in vivo) (e.g., a therapeutic protein) or a composition (e.g., a pharmaceutical composition composing the therapeutic agent (e.g., therapeutic protein)) to a subject, using any of the various methods and delivery systems known to those skilled in the art. Administering can also be performed, for example, once, a plurality of times, and/or over one or more extended periods. Therapeutic agents include agents whose effect is intended to be preventative (i.e., prophylactic).

The term “allogenic” with reference to the administration of a bodily substance (e.g., a population of cells (e.g., RBCs)) to a subject denotes that the bodily substance (e.g., a population of cells (e.g., RBCs)) was derived from a different subject than the subject to which the bodily substance is being or is to be administered to.

As used herein, the term “antibody” or “antibodies” is used in the broadest sense and encompasses various immunoglobulin (Ig) (e.g., human Ig (hIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments as defined herein). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In some embodiments, the antibody is a human, humanized, or chimeric IgG₁ or IgG₄ monoclonal antibody. In some embodiments, the term antibodies refers to a monoclonal or polyclonal antibody population.

As used herein, the term “immunogen” refers to a substance (e.g., a peptide or protein) that is capable of inducing an immune response (e.g., an adaptive immune response) in a subject (e.g., a human).

The term “autologous” with reference to the administration of a bodily substance (e.g., a population of cells (e.g., RBCs)) to a subject denotes that the bodily substance (e.g., population of cells (e.g., RBCs)) was derived from the same subject to which the bodily substance is being or is to be administered to.

As used herein, the term “auxotrophic” or “auxotroph” with respect to a Plasmodium refers to a Plasmodium that is incapable of synthesizing a particular organic compound required for its growth.

As used herein, the term “co-administering” and the like refers to the physical introduction of different therapeutic agents (or a precursor of the therapeutic agent that is metabolized or altered within the body of the subject to produce the therapeutic agent in vivo) (e.g., a therapeutic protein)) or different compositions (e.g., different pharmaceutical compositions each comprising a different therapeutic agent (e.g., a different therapeutic protein)) to the same subject. The different therapeutic agents or compositions (e.g., pharmaceutical compositions) may be administered simultaneously, at essentially the same time, or sequentially.

As used herein, the term “cognate compound” in reference to an auxotrophic Plasmodium refers to the organic compound (or a functional derivative, variant, fragment, or replacement thereof) that the auxotrophic Plasmodium is incapable of synthesizing but is needed for its growth.

As used herein, the term “CRISPR/Cas” refers to a genetic engineering system that comprises one or more gRNA and one or more RNA-guided endonuclease (e.g., nickase).

As used herein, the term “crRNA” refers to an RNA molecule (e.g., a gRNA or part of a gRNA molecule) that is capable of binding a protospacer in a target nucleic acid (e.g., DNA) molecule. In some embodiments, the crRNA comprises a scaffold portion that is not specific for a target nucleic acid molecule and a spacer portion that is specific for targeting to a target nucleic acid molecule. In some embodiments, the crRNA is capable of interacting with a nuclease (e.g., endonuclease) (e.g., a nuclease described herein (e.g., an endonuclease described herein)). In some embodiments, when the target nucleic acid (e.g., DNA) molecule is double stranded, the crRNA is capable of binding a protospacer within the strand opposite of the strand that contains a PAM. In some embodiments, the crRNA is sufficient for Cas nuclease to mediate nuclease activity. In some embodiments, the crRNA must be paired with a tracrRNA (e.g., as separate molecules or as a single gRNA) for a Cas nuclease to mediate nuclease activity.

As used herein, the term “derived from,” with reference to a polynucleotide refers to a polynucleotide that has at least 70% sequence identity to a reference polynucleotide (e.g., a naturally occurring polynucleotide) or a fragment thereof. The term “derived from,” with reference to a protein refers to a protein that comprises an amino acid sequence that has at least 70% sequence identity to the amino acid sequence of a reference protein (e.g., a naturally occurring protein). The term “derived from” as used herein does not denote any specific process or method for obtaining the polynucleotide, polypeptide, or protein. For example, the polynucleotide, polypeptide, or protein can be recombinant produced or chemically synthesized. In some embodiments, the polynucleotide or protein has at least 75% sequence identity to the reference polynucleotide or protein, respectively. In some embodiments, the polynucleotide or protein has at least 80% sequence identity to the reference polynucleotide or protein, respectively. In some embodiments, the polynucleotide or protein has at least 85% sequence identity to the reference polynucleotide or protein, respectively. In some embodiments, the polynucleotide or protein has at least 90% sequence identity to the reference polynucleotide or protein, respectively. In some embodiments, the polynucleotide or protein has at least 95% sequence identity to the reference polynucleotide or protein, respectively. In some embodiments, the polynucleotide or protein has at least 96% sequence identity to the reference polynucleotide or protein, respectively. In some embodiments, the polynucleotide or protein has at least 97% sequence identity to the reference polynucleotide or protein, respectively. In some embodiments, the polynucleotide or protein has at least 98% sequence identity to the reference polynucleotide or protein, respectively. In some embodiments, the polynucleotide or protein has at least 99% sequence identity to the reference polynucleotide or protein, respectively. In some embodiments, the polynucleotide or protein has 100% sequence identity to the reference polynucleotide or protein, respectively.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

As used herein the term “functional deletion” in reference to a gene refers to the deletion of enough of the nucleotide sequence of a protein coding gene such that the gene no longer expresses a functional protein. As such, a functional deletion of the gene includes both the entire deletion of a gene or a portion of the gene, as long as the edited gene no longer expresses a functional protein.

The term “functional variant” as used herein in reference to a protein refers to a protein that comprises at least one but no more than 15%, not more than 12%, no more than 10%, no more than 8% amino acid variation (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively reduced or eliminated. In some embodiments, the reference protein is a wild type protein.

The term “functional fragment” as used herein in reference to a protein refers to a fragment of a reference protein that retains at least one particular function. Not all functions of the reference protein need be retained by a functional fragment of the protein. In some instances, one or more functions are selectively reduced or eliminated. In some embodiments, the reference protein is a wild type protein.

As used herein, the term “fuse” and grammatical equivalents thereof refer to the operable connection of at least a first polypeptide to a second polypeptide, wherein the first and second polypeptides are not naturally found operably connected together. For example, the first and second polypeptides are derived from different proteins. The term fuse encompasses both a direct connection of the at least two polypeptides through a peptide bond, and the indirect connection through a linker (e.g., a peptide linker).

As used herein, the term “fusion protein” and grammatical equivalents thereof refers to a protein that comprises at least one polypeptide operably connected to another polypeptide, wherein the first and second polypeptides are not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “genome of a Plasmodium” and the like, refers to any genome of a Plasmodium, including the nuclear genome, mitochondrial genome, or apicoplast genome.

As used herein, the term “guide RNA” or “gRNA” refers to an RNA molecule that can associate with a nuclease (e.g., endonuclease) to direct the nuclease (e.g., endonuclease) to a target nucleic acid molecule (e.g., within a gene (e.g., within a cell)). In some embodiments, the gRNA comprises or consists of a crRNA. In some embodiments, the gRNA comprises a crRNA and a tracrRNA. As described throughout, in embodiments, wherein the gRNA comprises a crRNA and a tracrRNA, the crRNA and tracrRNA may be part of the same larger RNA molecule (e.g., a sgRNA) or separate RNA molecules.

As used herein, the term “heterologous” with reference to a nucleic acid molecule refers to a nucleic acid molecule that is not present within a larger nucleic acid molecule (e.g., a genome) in nature. For example, a nucleic acid molecule encoding a human cytokine inserted into the nuclear genome of a Plasmodium would be considered heterologous to the genome of a Plasmodium, as the nucleic acid molecule encoding the human cytokine is not found naturally in the nuclear genome of Plasmodia.

As used herein, the term “isolated” with reference to a polypeptide, protein, or polynucleotide refers to a polypeptide, protein, or polynucleotide that is substantially free of other cellular components with which it is associated in the natural state.

As used herein, the term “membrane protein” refers to a protein associated with the cellular membrane of a reference cell by which it is expressed and is at least exposed on the extracellular side of the cellular membrane.

As used herein, the term “non-pathogenic” with reference to a Plasmodium refers to a Plasmodium that is engineered such that it does not cause disease when administered to a subject. Auxotrophic Plasmodia described herein are non-pathogenic as the auxotrophic Plasmodia are incapable of growth in the absence of the cognate compound, as such appropriate co-administration of the cognate compound prevents the Plasmodium from being pathogenic in the subject. In some embodiments, the subject is mammal. In some embodiments, the subject is a human.

As used herein, the term “reference Plasmodium” and the like can refer to a non-engineered Plasmodium (e.g., does not comprise a heterologous nucleic acid molecule within a genome (e.g., encoding a therapeutic protein)).

The terms “nucleic acid molecule” and “polynucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “obtaining a sample” refers to the acquisition of a sample. The term includes the direct acquisition from a subject and the indirect acquisition through one or more third parties wherein one of the third parties directly acquired the sample from the subject.

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein).

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

As used herein, the terms “protein” and “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, the protein is folded into its three-dimensional structure. Where polypeptides are contemplated herein, it should be understood that proteins folded into their three-dimensional structure are also provided herein.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “RNA-guided endonuclease” refers to an endonuclease that utilizes a gRNA (e.g., described herein) within the system to target specific nucleic acid sequences for recognition.

As used herein, the term “sample” encompass a variety of biological specimens obtained from a subject. Exemplary sample types include, e.g., blood, red blood cells, and other liquid samples of biological origin (including, but not limited to, whole-blood, red blood cells (e.g., isolated from whole blood), peripheral blood mononuclear cells (PBMCs), serum, plasma, urine, saliva, amniotic fluid, stool, synovial fluid, etc.), nasopharyngeal swabs, solid tissue samples such as biopsies (or cells derived therefrom and the progeny thereof), tissue cultures (or cells derived therefrom and the progeny thereof), and cell cultures (or cells derived therefrom and the progeny thereof). The term also includes samples that have been manipulated in any way after their procurement from a subject, such as by centrifugation, filtration, washing, precipitation, dialysis, chromatography, lysis, treatment with reagents, enriched for certain cell populations, refrigeration, freezing, staining, etc. In some embodiments, the sample is a whole blood sample. In some embodiments, the sample is red blood cells (e.g., isolated from a whole blood sample).

As used herein, the term “signal peptide” refers to a sequence that can direct the transport or localization of a protein to a certain organelle, cell compartment, or extracellular export. The term encompasses both the signal sequence peptide and the nucleic acid sequence encoding the signal peptide. Thus, references to a signal peptide in the context of a nucleic acid molecule refers to the nucleic acid sequence encoding the signal peptide.

As used herein, the term “sgRNA” refers to a gRNA molecule that comprises both a crRNA and a tracrRNA. The components of the sgRNA may be arranged in any suitable order and any component may be operably connected to the adjacent component(s) directly or indirectly (e.g., via a nucleotide linker).

As used herein, the term “specifically binds” refers to preferential interaction, i.e., significantly higher binding affinity, between a first protein (e.g., a ligand) and a second protein (e.g., the ligand's cognate receptor) relative to other amino acid sequences. Herein, when a first protein is said to “specifically bind” to a second protein, it is understood that the first protein specifically binds to an epitope of the second protein. The term “epitope” refers to the portion of the second protein that the first protein specifically recognizes. The term specifically binds includes molecules that are cross reactive with the same epitope of a different species. For example, an antibody that specifically binds human Protein X may be cross reactive with Protein X of another species (e.g., cynomolgus, murine, etc.), and still be considered herein to specifically bind human Protein X.

As used herein, the term “subject” includes any animal, such as a human or other animal. In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “substantially complementary” with respect to two nucleotide sequences means a degree of complementarity sufficient for the nucleotide sequences to hybridize under standard stringent conditions. In some embodiments, the degree of complementarity of two nucleotide sequences is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides.

As used herein, the term “therapeutic protein” or “therapeutic polypeptide” refers to a protein that is intended for the preventing, treatment, and/or amelioration of a disease. The therapeutic protein may have a direct therapeutic effect or an indirect therapeutic effect. For example, the therapeutic protein may target a cell to a specific target (e.g., a target cell or tissue), wherein the cell (e.g., an agent, e.g., a protein secreted from the cell) mediates a direct therapeutic effect. The term also includes proteins intended for prophylactic (preventive) use (e.g., use in vaccines, etc.).

As used herein, the term “therapeutically effective amount” of a therapeutic agent (e.g., a therapeutic protein) refers to any amount of the therapeutic agent that, when used alone or in combination with another therapeutic agent, protects a subject against the onset of a disease (or infection) or promotes disease (or infection) regression evidenced by a decrease in severity of disease of infection symptoms, an increase in frequency and duration of disease or infection symptom-free periods, or a prevention of impairment or disability due to the disease or infection affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “tracrRNA” refers to an RNA molecule (e.g., part of a gRNA (e.g., a sgRNA)) that mediates binding of a gRNA to a nuclease (e.g., an endonuclease).

As used herein, the term “transmembrane protein” refers to a membrane protein that extends through the cellular membrane, with part of its mass on both the extracellular and intracellular sides of the membrane. Transmembrane proteins include for example, single and multi-pass transmembrane proteins.

As used herein, the terms “treat,” treating,” “treatment,” and the like refer to reducing, ameliorating, or preventing a disease or infection and/or symptom(s) associated therewith or obtaining a desired pharmacologic and/or physiologic effect. It will be appreciated that, although not precluded, treating a disease or infection does not require that the disease or infection, or symptom(s) associated therewith be completely eliminated or prevented. In some embodiments, the effect is therapeutic, i.e., without limitation, the effect partially or completely reduces, diminishes, abrogates, abates, alleviates, decreases the intensity of, or cures a disease and/or adverse symptom attributable to the disease or infection. In some embodiments, the effect is preventative, i.e., the effect protects or prevents an occurrence or reoccurrence of a disease or infection. To this end, the presently disclosed methods comprise administering a therapeutically effective amount of a compositions as described herein.

As used herein, the term “variant” or “variation” with reference to a polynucleotide, refers to a polynucleotide sequence that comprises at least one substitution, alteration, inversion, addition, or deletion of nucleotide compared to a reference polynucleotide sequence. As used herein, the term “variant” or “variation” with reference to a protein refers to a peptide or protein that comprises at least one substitution, alteration, inversion, addition, or deletion of an amino acid residue compared to a reference protein.

As used herein, the term “virulence protein” refers to a Plasmodium protein that functionally contributes to the pathogenicity of the Plasmodium in a host (e.g., a human subject).

### 5.2 Engineered Plasmodia

In one aspect, provided herein are engineered Plasmodia (or an engineered Plasmodium) comprising a heterologous nucleic acid molecule within a genome (e.g., the nuclear, mitochondrial, or apicoplastal genome) of the Plasmodia (or Plasmodium), wherein the heterologous nucleic acid molecule encodes a therapeutic protein (e.g., a therapeutic protein described herein).

Plasmodium is a genus of unicellular obligate intracellular parasites of vertebrates (e.g., mammals, e.g., humans) and insects. Plasmodium belongs to the phylum Apicomplexa, a taxonomic group of unicellular parasites with most containing a non-photosynthetic plastid called an apicoplast. Plasmodia have three organelles that contain their own genomic DNA, namely, the nucleus, the mitochondria, and the apicoplast. The apicoplast functions in part in the biosynthesis of metabolites including fatty acids, isoprenoids, and iron-sulphur clusters. See, e.g., Lim L, McFadden G I. The evolution, metabolism and functions of the apicoplast. Philos Trans R Soc Lond B Biol Sci. 2010; 365(1541):749-763. doi:10.1098/rstb.2009.0273. While over 100 species of Plasmodia have been identified, all Plasmodia share a similar vector-host lifecycle, wherein a Plasmodium infected vector (e.g., a mosquito) transmits sporozoites into the bloodstream of a host (e.g., a mosquito takes a blood meal injecting the sporozoites). The sporozoites migrate through the bloodstream to the liver where they form hepatic-stage schizonts, which rupture and release merozoites into the circulation. Once in the bloodstream the merozoites invade red blood cells (RBCs) where they mature to blood-stage schizonts. Some of the merozoites differentiate into male or female gametocytes, which can be ingested by a vector (e.g., a mosquito takes a blood meal ingesting the gametocytes). The sporozoites develop into sporozoites and complete the life cycle when the sporozoites are transmitted to a new host.

### 5.2.1 Plasmodia Species

Any Plasmodium species may be utilized. For example, any Plasmodium species that can infect humans (e.g., human cells) may be utilized. Exemplary Plasmodium species include, but are not limited to, Plasmodium falciparum (P. falciparum), Plasmodium vivax (P. vivax), Plasmodium malariae (P. malariae), Plasmodium ovale (P. ovale), and Plasmodium knowlesi (P. knowlesi). In some embodiments, the engineered Plasmodium is a P. falciparum, a P. malariae, a P. vivax, a P. ovale, or a P. knowlesi. In some embodiments, the engineered Plasmodium is a P. falciparum. Any suitable subspecies of any of the foregoing may also be utilized.

### 5.2.2 Pathogenic & Non-Pathogenic Plasmodia & Methods of Making

In some embodiments, the engineered Plasmodia are pathogenic or potentially pathogenic (e.g., in a subject (e.g., in a human)). In some embodiments, the engineered Plasmodia have not been modified to be attenuated and/or non-pathogenic (e.g., in humans). Pathogenic or potentially pathogenic Plasmodia may be utilized in certain methods described herein, e.g., in in vitro and ex vivo methods described herein.

In some embodiments, the engineered Plasmodia are non-pathogenic. Methods of making non-pathogenic Plasmodia are known in the art and examples described herein. For instance, non-pathogenic Plasmodia can be produced by engineering the Plasmodia to be auxotrophic or attenuated (e.g., as described herein). Non-pathogenic or attenuated engineered Plasmodia may be preferably utilized in certain methods described herein, e.g., in in vivo methods described herein.

### 5.2.2.1 Auxotrophic Plasmodia

Engineered Plasmodia described herein can be engineered to be auxotrophic (i.e., unable to synthesize one or more organic compounds essential for their growth). Methods of making auxotrophic Plasmodia are known in the art, see, e.g., Yeh E, DeRisi J L (2011) Chemical Rescue of Malaria Parasites Lacking an Apicoplast Defines Organelle Function in Blood-Stage Plasmodium falciparum. PLoS Biol 9(8): e1001138. https://doi.org/10.1371/journal.pbio.1001138 (hereinafter “Yeh 2011”), the full contents of which is incorporated by reference herein for all purposes. For example, the Plasmodium apicoplast relies on the prokaryotic non-mevalonate pathway for synthesis of isoprenoid precursors, such as isopentenyl pyrophosphate (IPP), which is essential for its survival (see, e.g., Yeh 2011). Therefore, chemical and/or genetic ablation of the apicoplast will generate an autotrophic Plasmodium dependent on IPP supplementation in vitro for survival. Moreover, as the levels of IPP in human blood serum are very low, chemical and/or genetic ablation of the apicoplast also renders the Plasmodium auxotrophic for IPP supplementation in vivo. As such, an IPP auxotrophic Plasmodium administered to a human subject will not be able to survive long term in vivo without the co-administration of IPP to the human subject.

As stated above, the apicoplast can be ablated through chemical and/or genetic means. For example, the apicoplast can be chemically ablated through exposure to an inhibitor of the non-mevalonate isoprenoid precursor biosynthesis pathway (e.g., fosmidomycin). In some embodiments, the apicoplast is chemically ablated through exposure of the Plasmodium to fosmidomycin. See, e.g., Yeh 2011. Alternatively, or in combination, the apicoplast can be ablated genetically, e.g., through the functional deletion of one or more genes that encode protein(s) essential for a functional apicoplast (See, e.g., Florentin A. et al. “Plastid biogenesis in malaria parasites requires the interactions and catalytic activity of the Clp proteolytic system,” PNAS, 117 (24) 13719-13729 (2020) https://doi.org/10.1073/pnas.1919501117, the entire contents of which is incorporated by reference herein for all purposes). For example, in some embodiments, the polyprenyl synthase (PPS) gene is functionally deleted. See, e.g., Megan Okada, Krithika Rajaram, Russell P Swift, Amanda Mixon, John Alan Maschek, Sean T Prigge, Paul A Sigala (2022) Critical role for isoprenoids in apicoplast biogenesis by malaria parasites eLife 11:e73208; https://doi.org/10.7554/eLife.73208 (hereinafter “Okada 2022”), the entire contents of which is incorporated by reference herein for all purposes). In some embodiments, one or more genes essential for the generation of IPP by the apicoplast are functionally and/or structurally deleted, such that the apicoplast does not produce IPP. For example, in some embodiments, the polyprenyl synthase (PPS) gene is functionally deleted. See, e.g., Okada 2020. The genetic engineering can be carried out and assessed using standard methods known in the art and described herein (see, e.g., § 5.3). For example, a CRISPR/Cas, TALEN, ZFN, and meganuclease system may be utilized (e.g., as described herein). These systems are standard in the art and can be optimized a person of ordinary skill in the art using routine experimentation (see, e.g., § 5.3), as well are in vitro assays to assess e.g., the success and efficiency of the genetic engineering.

### 5.2.2.2 Attenuated Plasmodia

Plasmodia can also be engineered to be non-pathogenic or attenuated through the functional deletion of one or more virulence genes. Non-pathogenic Plasmodia generated through this method are known in the art, see, e.g., Rostenberg, et al. A double-blind, placebo-controlled phase 1/2a trial of the genetically attenuated malaria vaccine PfSPZ-GA1. Science Translational Medicine, Vol 12:544 (May 20, 2020) DOI: 10.1126/scitranslmed.aaz5629 (hereinafter “Rostenberg 2020”), the full contents of which is incorporated by reference herein for all purposes. For example, in some embodiments, the engineered Plasmodium is a P. falciparum, wherein the virulence gene Plasmodium falciparum erythrocyte membrane protein 1 (PfEMP1) gene is functionally deleted. In some embodiments, the engineered Plasmodium comprises a functional deletion of one or more Var gene (e.g., PfEMP1). The genetic engineering can be carried out and assessed using standard methods known in the art and described herein (see, e.g., § 5.3). For example, CRISPR/Cas, TALEN, ZFN, and meganuclease systems may be utilized (e.g., as described herein). These systems are standard in the art and can be optimized a person of ordinary skill in the art using routine experimentation (see, e.g., § 5.3).

### 5.2.3 Heterologous Nucleic Acid Molecules

The heterologous nucleic acid molecule encoding the therapeutic protein can be positioned anywhere in the Plasmodium genome that allows for expression of the therapeutic protein. As discussed above, Plasmodia have three genomes: a nuclear genome, a mitochondrial genome, and an apicoplast genome. In some embodiments, the heterologous nucleic acid molecule encoding the therapeutic protein is positioned within the nuclear genome of the Plasmodium. In some embodiments, the heterologous nucleic acid molecule encoding the therapeutic protein is positioned within the mitochondrial genome of the Plasmodium. In some embodiments, the heterologous nucleic acid molecule encoding the therapeutic protein is positioned within the apicoplast genome of the Plasmodium. In one embodiment, the engineered Plasmodium is a P. falciparum, and the heterologous nucleic acid molecule is positioned within the Pf47 gene of the nuclear genome. See, e.g., Talman, A. M., Blagborough, A. M., & Sinden, R. E. (2010). A Plasmodium falciparum strain expressing GFP throughout the parasite's life-cycle. PloS one, 5(2), e9156. https://doi.org/10.1371/journal.pone.0009156 (hereinafter “Talman 2010”), the entire contents of which is incorporated by reference herein for all purposes. In one embodiment, the heterologous nucleic acid molecule is positioned within the HAP110 gene of the nuclear genome of the Plasmodium. See, e.g., Florentin A. et al, “The Clp System in Malaria Parasites Degrades Essential Substrates to Regulate Plastid Biogenesis” bioRxiv 718452 (2019); doi: https://doi.org/10.1101/718452, the entire contents of which is incorporated by reference herein for all purposes). In one embodiment, the engineered Plasmodium is a P. falciparum, and the heterologous nucleic acid molecule encoding the therapeutic protein is positioned within the HAP110 gene of the nuclear genome. In some embodiments, the heterologous nucleic acid molecule further encodes a signal peptide (see, e.g., § 5.2.4).

In some embodiments, the nucleic acid molecule is a DNA nucleic acid molecule. In some embodiments, the heterologous nucleic acid molecule (e.g., the DNA nucleic acid molecule) is codon optimized. Codon optimization, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.2.4 Therapeutic Proteins & Polypeptides

The therapeutic protein can be any therapeutic protein that is capable of being expressed by the engineered Plasmodia described herein. In some embodiments, the therapeutic protein is a secreted protein, an intracellular protein (e.g., an enzyme), or a membrane protein (e.g., a transmembrane protein). In some embodiments, the membrane protein is a transmembrane protein. In some embodiments, the therapeutic protein is an antibody, an enzyme, a cytokine, a chemokine, a hormone, a growth factor, a fusion protein (e.g., an antibody cytokine fusion protein), or an immunogen, or a functional fragment or functional variant of any of the foregoing. In some embodiments, the therapeutic protein is an immunogenic protein, e.g., a vaccine, part of a vaccine.

In some embodiments, the therapeutic protein is an antibody. In some embodiments, the therapeutic protein is an enzyme. In some embodiments, the therapeutic protein is a cytokine. In some embodiments, the therapeutic protein is a chemokine. In some embodiments, the therapeutic protein is a hormone. In some embodiments, the therapeutic protein is a growth factor. In some embodiments, the therapeutic protein is a fusion protein (e.g., an antibody cytokine fusion protein). In some embodiments, the therapeutic protein is an immunogen.

In some embodiments, the therapeutic protein is an immune cell inhibitor (e.g., a T cell inhibitor, a B cell, an NK cell inhibitor, a T regulatory cell inhibitor, a Th17 cell inhibitor, a dendritic cell inhibitor, a macrophage inhibitor). In some embodiments, the therapeutic protein is a cytokine inhibitor. In some embodiments, the therapeutic protein is a chemokine inhibitor. In some embodiments, the therapeutic protein is an interleukin inhibitor.

In some embodiments, the therapeutic protein is an IL-1, IL-2, IL-2, IL-4, IL-5, IL-6, IL-7, IL-8, -IL9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, IL-39, and/or IL-40 inhibitor. In some embodiments, the therapeutic protein is an IFNα, IFNβ, IFNγ, TNFα, TNFβ, LT-β, cd154, GMCSF, GCSF, CSIF, MCSF, MSP, SCF, NGF, MCP-1, LIF, OSM, 4-1BBL, APRIL, CD70, CD153, CD178, GITRL, LIGHT, OX40L, TALL-1, TRAIL, TWEAK, TRANCE, TGFβ1, TGFβ2, TGFβ3, Flts-3L, TPO, EPO, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL1, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CCR1, CCR2, CCR3, CCR4, CCR5, CCR6, CCR7, CCR8, CCR9, CCR10, CXCR1, CXCR2, CXCR3, CXCR4, CXCR5, or CXCR6 inhibitor.

In some embodiments, the therapeutic protein (e.g., an enzyme) is protected from the host subject's immune system inside the Plasmodia-infected RBC of the subject. In embodiments, the therapeutic protein is immunogenic if administered to the subject directly, e.g., the therapeutic protein is not naturally produced by the subject. In embodiments, the therapeutic protein not naturally occurring in the subject is an enzyme not naturally occurring in the subject, e.g., uricase or asparaginase. In one embodiment, the target substrate (e.g., uric acid) diffuses from the blood into the RBCs which where it can be broken down by the therapeutic enzyme (e.g., uricase), in a subject suffering from gout. In another embodiment, the target substrate (e.g., asparagine) diffuses from the blood into the RBCs which where it can be broken down by the therapeutic enzyme (e.g., asparaginase), in a subject suffering from a blood cancer, e.g., acute lymphoblastic leukemia (ALL).

In some embodiments, the therapeutic protein functions to target an RBC expressing the therapeutic protein to a target cell or tissue (e.g., in vivo). In some embodiments, the therapeutic protein is a membrane (e.g., transmembrane) protein that specifically binds a membrane (e.g., transmembrane) protein of a target cell (e.g., in vivo). In some embodiments, the therapeutic protein is a membrane (e.g., transmembrane) protein that specifically binds a membrane (e.g., transmembrane) protein of a target cell (e.g., in vivo), to thereby target the RBC expressing the therapeutic protein to a target cell or tissue (e.g., in vivo).

In some embodiments, the therapeutic protein further comprises a signal peptide. Signal peptides for use in therapeutic proteins are commonly known in the art and can be selected by a person of ordinary skill in the art using standard methodology known in the art. For example, commonly used signal peptides include the native signal peptide (or functional variant or functional fragment thereof) of human interleukin 2 (hJL-2), human oncostatin M (hOSM), human chymotrypsinogen (hCTRB1), human trypsinogen 2 (hTRY2), and human insulin (hINS).

Exemplary therapeutic proteins include, but are not limited to, abatacept, adalimumab, adalimumab-atto, certolizumab, certolizumab pegol, etanercept, etanercept-szzs, golimumab, golimumab injection, infliximab, infliximab-dyyb, secukinumab, tocilizumab, ustekinumab, anakinra, canakinumab, rituximab, tocilizumab, interferon beta-1b, daclizumab, interferon beta-1a, natalizumab, interferon beta-1a, peginterferon beta-1a, ixekizumab, brodalumab, bimekizumab, tildrakizumab, tildrakizumab-asmn, risankizumab, guselkumab, rilonacept, belimumab, interferon gamma-1b, abciximab, alteplase, reteplase, tenecteplase, abobotulinum toxin a, onabotulinum toxin a, incobotulinum toxin a, anakinra, rimabotulinum toxin b, follitropin alpha, abobotulinum toxin a, onabotulinum toxin a, collagenase, ecallantide, collagenase Clostridium histolyticum, aflibercept, ziv-aflibercept, ranibizumab, ocriplasmin, bevacizumab, pegaptanib, interferon alfa-2b, obinutuzumab, asparaginase, asparaginase Erwinia chrysanthemi, blinatumomab, interferon alfa-2b, obinutuzumab, ofatumumab, pegaspargase, sargramostim, nivolumab, brentuximab vedotin, ibritumomab tiuxetan, rituximab, ado-trastuzumab emtansine, pertuzumab, trastuzumab, aldesleukin, daratumumab, elotuzumab, pembrolizumab, ipilimumab, atezolizumab, cetuximab, atezolizumab, necitumumab, ramucirumab, panitumumab, capromab pendetide, olaratumab, elosulfase alfa, idursulfase, iaronidase, asfotase alfa, pegloticase, alirocumab, evolocumab, albiglutide, dulaglutide, becaplermin, agalsidase beta, alglucosidase alfa, canakinumab, galsulfase, metreleptin, rasburicase, sebelipase alfa, parathyroid hormone, bezlotoxumab, interferon alfa-2b, interferon alfa-n3, peginterferon alfa-2a, peginterferon alfa-2b, palivizumab, siltuximab, pegfilgrastim, sargramostim, epoetin alfa, methoxy polyethylene glycol-epoetin beta, basiliximab, belatacept, sargramostim, palifermin, eculizumab, oprelvekin, romiplostim, glucarpidase, idarucizumab, obiltoxaximab, raxibacumab, dornase alfa, mepolizumab, reslizumab, and omalizumab.

A list of exemplary therapeutic proteins are provided in Table 1 below. The therapeutic proteins listed in Table 1 are exemplary and not intended to be limiting.

TABLE 1
Exemplary Therapeutic Proteins.

| Description | Exemplary Indication(s) |
|---|---|
| Abatacept | Arthritis |
| | Psoriatic Arthritis |
| Adalimumab | Arthritis |
| | Ankylosing Spondylitis |
| | Psoriasis |
| | Psoriatic Arthritis |
| | Crohn's Disease |
| | Ulcerative Colitis |
| Adalimumab-Atto | Arthritis |
| | Ankylosing Spondylitis |
| | Psoriasis |
| | Psoriatic Arthritis |
| | Crohn's Disease |
| | Ulcerative Colitis |
| Certolizumab | Psoriatic Arthritis |
| Certolizumab Pegol | Arthritis |
| | Ankylosing Spondylitis |
| | Psoriatic Arthritis |
| Etanercept | Arthritis |
| | Ankylosing Spondylitis |
| | Psoriatic Arthritis |
| Etanercept-Szzs | Arthritis |
| | Ankylosing Spondylitis |
| | Psoriatic Arthritis |
| Golimumab | Arthritis |
| | Ankylosing Spondylitis |
| | Ulcerative Colitis |
| Golimumab Injection | Arthritis |
| | Ankylosing Spondylitis |
| | Ulcerative Colitis |
| Infliximab | Arthritis |
| | Ankylosing Spondylitis |
| | Psoriasis |
| | Psoriatic Arthritis |
| | Crohn's Disease |
| | Ulcerative Colitis |
| Infliximab-Dyyb | Arthritis |
| | Ankylosing Spondylitis |
| | Psoriasis |
| | Psoriatic Arthritis |
| | Crohn's Disease |
| | Ulcerative Colitis |
| Secukinumab | Arthritis |
| | Ankylosing Spondylitis |
| | Psoriasis |
| | Psoriatic Arthritis |
| Tocilizumab | Arthritis |
| Ustekinumab | Arthritis |
| | Psoriasis |
| | Psoriatic Arthritis |
| | Crohn's Disease |
| Anakinra | Arthritis |
| Canakinumab | Arthritis |
| Rituximab | Arthritis |
| Tocilizumab | Arthritis |
| Interferon Beta-1b | Multiple Sclerosis |
| Daclizumab | Multiple Sclerosis |
| Interferon Beta-1a | Multiple Sclerosis |
| Natalizumab | Multiple Sclerosis |
| Interferon Beta-1a | Multiple Sclerosis |
| Peginterferon Beta-1a | Multiple Sclerosis |
| Ixekizumab | Psoriasis |
| | Psoriatic Arthritis |
| Brodalumab | Psoriasis |
| | Psoriatic Arthritis |
| Bimekizumab | Psoriatic Arthritis |
| Tildrakizumab | Psoriatic Arthritis |
| Tildrakizumab-Asmn | Psoriatic Arthritis |
| Risankizumab | Psoriatic Arthritis |
| Guselkumab | Psoriatic Arthritis |
| Rilonacept | Pericarditis |
| | Cryopyrin-Associated Periodic Syndromes |
| Belimumab | Systemic Lupus Erythematosus |
| Interferon Gamma-1b | Chronic Granulomatous Disease |
| Abciximab | Myocardial Ischemia |
| Alteplase | Myocardial Infarction |
| Reteplase | Myocardial Infarction |
| Tenecteplase | Myocardial Infarction |
| Abobotulinum Toxin A | Neurological Conditions |
| Onabotulinum Toxin A | Neurological Conditions |
| Incobotulinum Toxin A | Neurological Conditions |
| Anakinra | Neurological Conditions |
| Rimabotulinum Toxin B | Neurological Conditions |
| Follitropin Alpha | Neurological Conditions |
| Abobotulinum Toxin A | Dermatological Conditions |
| Onabotulinum Toxin A | Dermatological Conditions |
| Collagenase | Dermatological Conditions |
| Ecallantide | Dermatological Conditions |
| Incobotulinum Toxin A | Dermatological Conditions |
| Collagenase Clostridium | Dermatological Conditions |
| Histolyticum | |
| Aflibercept | Eye Conditions; Cancer (e.g., Colorectal) |
| Ziv-Aflibercept | Eye Conditions; Cancer (e.g., Colorectal) |
| Ranibizumab | Eye Conditions |
| Incobotulinum Toxin A | Eye Conditions |
| Ocriplasmin | Eye Conditions |
| Bevacizumab | Eye Conditions; Cancer (e.g., Colorectal) |
| Pegaptanib | Eye Conditions |
| Interferon Alfa-2b | Cancer (e.g., Leukemia, Lymphoma) |
| Obinutuzumab | Cancer (e.g., Leukemia) |
| Asparaginase | Cancer (e.g., Leukemia) |
| Asparaginase Erwinia | Cancer (e.g., Leukemia) |
| Chrysanthemi | |
| Blinatumomab | Cancer (e.g., Leukemia) |
| Interferon Alfa-2b | Cancer (e.g., Leukemia, Lymphoma, Skin (e.g., |
| | Melanoma)) |
| Obinutuzumab | Cancer (e.g., Leukemia, Lymphoma) |
| Ofatumumab | Cancer (e.g., Leukemia) |
| Pegaspargase | Cancer (e.g., Leukemia) |
| Sargramostim | Cancer (e.g., Leukemia) |
| Nivolumab | Cancer (e.g., Lymphoma, Skin (e.g., Melanoma), |
| | Carcinoma, Lung) |
| Brentuximab Vedotin | Cancer (e.g., Lymphoma) |
| Ibritumomab Tiuxetan | Cancer (e.g., Lymphoma) |
| Rituximab | Cancer (e.g., Lymphoma) |
| Ado-Trastuzumab Emtansine | Cancer (e.g., Breast) |
| Pertuzumab | Cancer (e.g., Breast) |
| Trastuzumab | Cancer (e.g., Breast, Gastric) |
| Aldesleukin | Cancer (e.g., Skin, (e.g., Melanoma), Carcinoma) |
| Daratumumab | Cancer (e.g., Skin, e.g., Melanoma) |
| Elotuzumab | Cancer (e.g., Skin, e.g., Melanoma) |
| Pembrolizumab | Cancer (e.g., Skin, (e.g., Melanoma), Lung) |
| Ipilimumab | Cancer (e.g., Skin, (e.g., Melanoma), Carcinoma) |
| Atezolizumab | Cancer (e.g., Carcinoma, Lung) |
| Cetuximab | Cancer (e.g., Carcinoma, Colorectal) |
| Atezolizumab | Cancer (e.g., Lung) |
| Necitumumab | Cancer (e.g., Lung) |
| Ramucirumab | Cancer (e.g., Lung, Colorectal, Gastric) |
| Panitumumab | Cancer (e.g., Colorectal) |
| Capromab Pendetide | Cancer (e.g., Prostate) |
| Olaratumab | Cancer (e.g., Solid Tumors) |
| Elosulfase Alfa | Mucopolysaccharidosis |
| Idursulfase | Mucopolysaccharidosis |
| Iaronidase | Mucopolysaccharidosis |
| Asfotase Alfa | Hypophosphatasia |
| Pegloticase | Gout |
| Alirocumab | Hypercholesterolemia |
| Evolocumab | Hypercholesterolemia |
| Albiglutide | Diabetes |
| Dulaglutide | Diabetes |
| Becaplermin | Diabetes |
| Agalsidase Beta | Fabry disease |
| Alglucosidase Alfa | Pompe disease |
| Canakinumab | Cryopyrin-Associated Periodic Syndromes; Systemic |
| | Juvenile Idiopathic Arthritis |
| Galsulfase | Mucopolysaccharidosis VI |
| Metreleptin | Lipodystrophy |
| Rasburicase | Hyperuricemia |
| Sebelipase Alfa | Lysosomal Acid Lipase Deficiency |
| Parathyroid Hormone | Hypocalcemia |
| Bezlotoxumab | Infection (e.g., Bacterial) |
| Interferon Alfa-2b | Infection (e.g., Viral) |
| Interferon Alfa-N3 | Infection (e.g., Viral) |
| Peginterferon Alfa-2a | Infection (e.g., Viral) |
| Peginterferon Alfa-2b | Infection (e.g., Viral) |
| Palivizumab | Infection (e.g., Viral) |
| Siltuximab | Infection (e.g., Viral) |
| Pegfilgrastim | Neutropenia |
| Sargramostim | Neutropenia |
| Epoetin Alfa | Anemia |
| Methoxy Polyethylene Glycol- | Anemia |
| Epoetin Beta | |
| Basiliximab | Transplant Rejection |
| Belatacept | Transplant Rejection |
| Sargramostim | Transplant Rejection |
| Palifermin | Transplant Rejection |
| Eculizumab | Paroxysmal Nocturnal Hemoglobinuria; Atypical |
| | Hemolytic Uremic Syndrome |
| Oprelvekin | Thrombocytopenia |
| Romiplostim | Thrombocytopenia |
| Glucarpidase | Methotrexate Toxicity |
| Idarucizumab | Dabigatran Reversal |
| Obiltoxaximab | Inhalational Anthrax |
| Raxibacumab | Inhalational Anthrax |
| Dornase Alfa | Cystic Fibrosis |
| Mepolizumab | Eosinophilic Asthma |
| Reslizumab | Eosinophilic Asthma |
| Omalizumab | Eosinophilic Asthma |

In some embodiments, the therapeutic protein is a therapeutic protein listed in Table 1. In some embodiments, the therapeutic protein comprises a therapeutic protein listed in Table 1. In some embodiments, the therapeutic protein consists of a therapeutic protein listed in Table 1.

In some embodiments, the therapeutic protein can be used treat a disease. In some embodiments, the therapeutic protein can be used treat a proinflammatory disease, an autoimmune disease, an infection, a cancer, a neurological condition, an eye condition, a dermatological condition, transplant rejection, a blood disorder, a cardiovascular disease, a metabolic disease, bone disease, an endocrine disease, a genetic disease, a lipid disease, a uric acid disease, a calcium disease, or a drug toxicity.

Exemplary pro-inflammatory diseases include, but are not limited to, arthritis, rheumatoid arthritis, psoriasis, psoriatic arthritis, Crohn's disease, ulcerative colitis, ankylosing spondylitis, multiple sclerosis, pericarditis, cryopyrin-associated periodic syndromes, systemic lupus erythematosus, chronic granulomatous disease, gout, systemic juvenile idiopathic arthritis, transplant rejection, diabetes, asthma, and eosinophilic asthma.

Exemplary autoimmune diseases include, but are not limited to, multiple sclerosis, Crohn's disease, ulcerative colitis, systemic lupus erythematosus, diabetes, rheumatoid arthritis, psoriasis, psoriatic arthritis, ankylosing spondylitis, arthritis, autoimmune pericarditis, Cryopyrin-associated autoinflammatory syndromes (CAPS), and autoimmune diabetes mellitus.

Cancers include, e.g., solid tumors and liquid cancers. Exemplary cancers include, but are not limited to, colon cancer, rectal cancer, colorectal cancer, skin cancer (e.g., melanoma), lung cancer, breast cancer, gastric cancer, leukemia, and lymphoma. Exemplary metabolic diseases include, but are not limited to, mucopolysaccharidosis.

Exemplary cardiovascular diseases include, but are not limited to, myocardial ischemia, myocardial infarction, and hypercholesterolemia. Exemplary bone diseases include, but are not limited to, hypophosphatasia. Exemplary endocrine diseases include, but are not limited to, diabetes. Exemplary neurological diseases include, but are not limited to, Fabry disease. Exemplary genetic disease include, but are not limited, to, Pompe disease, mucopolysaccharidosis VI, lysosomal acid lipase deficiency, atypical hemolytic uremic syndrome, cystic fibrosis, and Fabry disease. Exemplary lipid diseases include, but are not limited to, lipodystrophy, and cystic fibrosis, Exemplary uric acid diseases include, but are not limited to, hyperuricemia. Exemplary calcium diseases include, but are not limited to, hypocalcemia. Exemplary infections include, but are not limited to, viral infections, bacterial infections, parasite infections, yeast infections, and fungal infections. Exemplary blood disorders include, but are not limited to, neutropenia, anemia, paroxysmal nocturnal hemoglobinuria, atypical hemolytic uremic syndrome, and thrombocytopenia. Exemplary drug toxicities include, but are not limited to, methotrexate toxicity and dabigatran toxicity.

### 5.3 Methods of Making Engineered Plasmodia

Further provided herein are methods of making an engineered Plasmodium (or a population of engineered Plasmodia) described herein. In one aspect, provided herein are methods of making an engineered Plasmodium (e.g., an engineered Plasmodium described herein) (or a population of engineered Plasmodia described herein) comprising: culturing a reference Plasmodium (or reference population of Plasmodia); genetically modifying the reference Plasmodium (or the reference population of Plasmodia) to introduce a heterologous nucleic acid molecule encoding a therapeutic protein (e.g., a therapeutic protein described herein) into a genome of the reference Plasmodium (or the population of reference Plasmodia); to thereby make an engineered Plasmodium (e.g., an engineered Plasmodium described herein) (or a population of engineered Plasmodia described herein).

The reference Plasmodium or population of Plasmodia can be cultured in any suitable culture medium. Standard culture mediums for in vitro culture of Plasmodia are known in the art, see, e.g., Talman 2010 (describing the culture of P. falciparum 3D7 in 4% haematocrit RPMI 1640 (Gibco, UK), supplemented with 10% human AB serum, and gassed with 5% CO2, 0.5% O2 in N2 at 37° C.); and Hoppe H C, Verschoor J A, Louw A I. Plasmodium falciparum: a comparison of synchronisation methods for in vitro cultures. Exp Parasitol. 1991 May; 72(4):464-7. doi: 10.1016/0014-4894(91)90093-c. PMID: 1902796 (hereinafter “Hoppe 1991”) (describing the culture of P. falciparum 3D7 parasites in a complete medium at 1% haematocrit at 37° C. in a 5% CO₂/3% O₂/balanced N₂ gas mixture), Schuster F L. Cultivation of Plasmodium spp. Clin Microbiol Rev. 2002; 15(3):355-364. doi:10.1128/CMR.15.3.355-364.2002 (hereinafter “Schuster 2002”); the full contents of each of which is incorporated by reference herein for all purposes. The Examples provided herein (e.g., Example 4) further provide exemplary suitable culture media.

In some embodiments, the culturing step comprises culturing the reference Plasmodium (or population of Plasmodia) in a composition comprising human RBCs (hRBCs). The composition comprising hRBCs can be for example, a whole human blood sample or hRBCs isolated from a whole human blood sample (e.g., diluted in a suitable buffer, e.g., RPMI (see, e.g., Talman 2010, Hoppe 1991). The hRBCs may be A+, A−, B+, B−, O+, O−, AB+, or AB−. In some embodiments, the hRBCs are O−. Standard methods of obtaining, handling, storing, and using hRBCs (e.g., hRBC samples, e.g., hRBC samples obtained from whole human blood samples) are known in the art, see, e.g., Childs, R. A., Miao, J., Gowda, C. et al. An alternative protocol for Plasmodium falciparum culture synchronization and a new method for synchrony confirmation. Malar J 12, 386 (2013). https://doi.org/10.1186/1475-2875-12-386 (hereinafter “Childs 2013”), the full contents of which is incorporated by reference herein. For example, typically hRBCs isolated from a whole human blood sample will be e.g., washed prior to use and if stored prior to use—stored at 4-8° C. for use within several weeks (e.g., 3 weeks). See, e.g., Childs 2013.

In some embodiments, the parasitemia (i.e., the quantitative content of Plasmodia in the culture of hRBCs) is monitored (e.g., by preparing blood films from a sample of the culture, staining with Giemsa stain following methanol fixation, and counting infected hRBCs microscopically, see, e.g., Schuster 2011). In some embodiments, the parasitemia of the culture (e.g., the initial culture) is from about 0.1% to about 5%. In some embodiments, the parasitemia of the culture (e.g., the initial culture) is at least about 0.1%, 0.5%, 1%, 2%, 3%, 4%, or 5%. In some embodiments, the parasitemia of the culture (e.g., the initial culture) is about 0.1%, 0.5%, 1%, 2%, 3%, 4%, or 5%. In some embodiments, the parasitemia of the culture (e.g., the initial culture) is no more than 0.1%, 0.5%, 1%, 2%, 3%, 4%, or 5%. In some embodiments, the parasitemia is maintained at substantially the same value over the course of the culture. In some embodiments, the parasitemia is changed during the course of the culture (e.g., the initial parasitemia is lower and it increases over the course of the culture).

In some embodiments, the hematocrit (i.e., the percentage of hRBCs in the culture) is monitored. In some embodiments, the hematocrit of the culture is from about 0.5% to about 5%. In some embodiments, the hematocrit of the culture is at least about 0.1%, 0.5%, 1%, 2%, 3%, 4%, or 5%. In some embodiments, the hematocrit of the culture is about 0.1%, 0.5%, 1%, 2%, 3%, 4%, or 5%. In some embodiments, the hematocrit of the culture is no more than 0.1%, 0.5%, 1%, 2%, 3%, 4%, or 5%. In some embodiments, the hematocrit of the culture is about 2%. In some embodiments, the hematocrit of the culture is maintained at substantially the same value over the course of the culture. In some embodiments, the hematocrit of the culture is changed during the course of the culture.

In some embodiments, the method comprises isolating the reference Plasmodium (or population of reference Plasmodia) from the hRBCs prior to genetically modifying the reference Plasmodium (or population of reference Plasmodia). Viable Plasmodia can be isolated from hRBCs using standard methods in the art, e.g., detergent (e.g., saponin) mediated lysis of the hRBCs. See, e.g., Mutungi Joe et al (2015) Isolation of invasive Plasmodium yoelii merozoites with a long half-life to evaluate invasion dynamics and potential invasion inhibitors. Molecular and Biochemical Parasitology; doi:10.1016/j.molbiopara.2015.12.003. 10.1016/j.molbiopara.2015.12.003 (hereinafter “Mutungi 2015”); Radfar, A., Méndez, D., Moneriz, C. et al. Synchronous culture of Plasmodium falciparum at high parasitemia levels. Nat Protoc 4, 1899-1915 (2009). https://doi.org/10.1038/nprot.2009.198 (hereinafter “Radfar 2009”); the full contents of each of which are incorporated by reference herein for all purposes.

In some embodiments, the method further comprises synchronizing the population of Plasmodia to a single developmental stage. Standard methods of synchronization are known in the art and include for example Percoll/sorbitol synchronization and Percoll density centrifugation; see, e.g., Hoppe 1991, Childs 2013, and Schuster 2011. In some embodiments, the Plasmodia are synchronized prior to the genetic modification.

In some embodiments, the engineered Plasmodium or (population of engineered Plasmodia) are isolated/purified from the cultured hRBCs. Standard methods are known in the art to isolating/purifying Plasmodium from hRBCs utilizing a detergent (e.g., saponin) to lyse the hRBCs, see, e.g., Mutungi 2015, Radfar 2009. Any isolation method may be used such that viable Plasmodia are obtained. The standard methods can be optimized by a person of ordinary skill in the art using routine experimentation.

In some embodiments, the engineered Plasmodium or population of engineered Plasmodia (or a sample of the population of engineered Plasmodia) is assessed for viability. Standard assays to assess Plasmodium viability are known in the art, including, e.g., microscopy (e.g., a morphological assessment), exflagellation assays, experimental infection in a vector host (e.g., an insect), and an RBC invasion assay (e.g., by flow cytometry), see, e.g., Ramos G et al. Front. Cell. Infect. Microbiol., Vol. 11, 1 Jun. 2021 https://doi.org/10.3389/fcimb.2021.676276 (hereinafter “Ramos”); Lelliott, P. M., Lampkin, S., McMorran, B. J. et al. A flow cytometric assay to quantify invasion of red blood cells by rodent Plasmodium parasites in vivo. Malar J 13, 100 (2014), https://doi.org/10.1186/1475-2875-13-100 (hereinafter “Lelliott 2014”); the full contents of each of which is incorporated by reference herein for all purposes.

The heterologous nucleic acid molecule can be inserted into any amendable gene locus in the Plasmodium genome. Exemplary gene loci can be found at https://plasmodb.org/plasmo/app/ (the entire contents of which are incorporated herein by reference for all purposes). Exemplary preferred gene loci are set forth in Table 2.

TABLE 2
Exemplary Target Genetic Loci for Heterologous
Nucleic Acid Molecule Integration.

| Gene ID* | Gene Name | Genomic Location |
|---|---|---|
| PF3D7_1462800 | GAPDH | Pf3D7_14_v3: 2,558,055 . . . 2,560,979(+) |
| PF3D7_0818900 | HSP70 | Pf3D7_08_v3: 858,550 . . . 862,980(+) |
| PF3D7_0500800 | MESA | Pf3D7_05_v3: 42,114 . . . 48,258(−) |
| PF3D7_1228600 | MSP9 | Pf3D7_12_v3: 1,160,654 . . . 1,163,264(−) |
| PF3D7_1012400 | HGPRT | Pf3D7_10_v3: 474,731 . . . 477,029(−) |
| PF3D7_1015900 | ENO | Pf3D7_10_v3: 636,671 . . . 639,498(+) |
| PF3D7_0708400 | HSP90 | Pf3D7_07_v3: 380,449 . . . 385,546(+) |
| PF3D7_1343000 | PMT | Pf3D7_13_v3: 1,695,462 . . . 1,699,267(+) |
| PF3D7_1105100 | H2B | Pf3D7_11_v3: 225,227 . . . 226,943(+) |
| PF3D7_1324900 | LDH | Pf3D7_13_v3: 1,040,006 . . . 1,042,682(+) |
| PF3D7_1444800 | FBPA | Pf3D7_14_v3: 1,843,368 . . . 1,846,989(−) |
| PF3D7_1016300 | GBP130 | Pf3D7_10_v3: 648,012 . . . 654,565(−) |
| PF3D7_1246200 | ACT1 | Pf3D7_12_v3: 1,919,898 . . . 1,923,323(+) |
| PF3D7_1136500 | CK1 | Pf3D7_11_v3: 1,431,881 . . . 1,435,170(−) |
| PF3D7_1136500 | CK1 | Pf3D7_11_v3: 1,431,881 . . . 1,435,170(−) |
| PF3D7_0626800 | PyrK | Pf3D7_06_v3: 1,078,020 . . . 1,082,073(+) |
| PF3D7_1347200 | NT1 | Pf3D7_13_v3: 1,889,277 . . . 1,891,491(−) |
| PF3D7_0731500 | EBA175 | Pf3D7_07_v3: 1,357,251 . . . 1,363,653(+) |
| PF3D7_0813300 | N/A | Pf3D7_08_v3: 655,382 . . . 658,303(+) |
| PF3D7_0202500 | ETRAMP2 | Pf3D7_02_v3: 127,203 . . . 129,089(+) |
| PF3D7_0814200 | ALBA1 | Pf3D7_08_v3: 684,997 . . . 689,981(+) |
| PF3D7_0917900 | BIP | Pf3D7_09_v3: 736,669 . . . 741,656(+) |
| PF3D7_1027300 | nPrx | Pf3D7_10_v3: 1,139,669 . . . 1,144,013(−) |
| PF3D7_0516900 | RPL2 | Pf3D7_05_v3: 712,371 . . . 714,307(−) |
| PF3D7_0930300 | MSP1 | Pf3D7_09_v3: 1,201,305 . . . 1,207,576(+) |
| PF3D7_0102200 | RESA | Pf3D7_01_v3: 97,590 . . . 103,615(+) |
| PF3D7_1149200 | RESA3 | Pf3D7_11_v3: 1,975,705 . . . 1,980,325(+) |
| PF3D7_1102800 | ETRAMP11.2 | Pf3D7_11_v3: 129,265 . . . . . . 132,824(−) |
| PF3D7_1468700 | eIF4A | Pf3D7_14_v3: 2,824,250 . . . 2,826,936(+) |
| PF3D7_0532100 | ETRAMP5 | Pf3D7_05_v3: 1,300,188 . . . 1,302,740(+) |
| PF3D7_0714000 | H2B.Z | Pf3D7_07_v3: 642,200 . . . 643,997(−) |
| PF3D7_1011800 | PREBP | Pf3D7_10_v3: 452,765 . . . 459,985(+) |
| PF3D7_1103100 | RPP1 | Pf3D7_11_v3: 139,700 . . . 141,251(−) |
| PF3D7_0827900 | PDI8 | Pf3D7_08_v3: 1,206,438 . . . 1,209,016(−) |
| PF3D7_1121600 | EXP1 | Pf3D7_11_v3: 814,343 . . . 818,094(−) |
| PF3D7_0905400 | RhopH3 | Pf3D7_09_v3: 270,371 . . . 275,833(−) |
| PF3D7_1117700 | RAN | Pf3D7_11_v3: 670,173 . . . 673,050(−) |
| PF3D7_1453700 | P23 | Pf3D7_14_v3: 2,206,046 . . . 2,209,273(+) |
| PF3D7_1038000 | N/A | Pf3D7_10_v3: 1,504,966 . . . 1,507,544(+) |
| PF3D7_1038000 | N/A | Pf3D7_10_v3: 1,504,966 . . . 1,507,544(+) |
| PF3D7_0513300 | PNP | Pf3D7_05_v3: 568,594 . . . 570,527(−) |
| PF3D7_0919000 | NAPS | Pf3D7_09_v3: 780,844 . . . 783,347(−) |
| PF3D7_0721600 | N/A | Pf3D7_07_v3: 930,961 . . . 933,016(−) |
| PF3D7_0525800 | IMC1g | Pf3D7_05_v3: 1,071,094 . . . 1,072,988(+) |
| PF3D7_1317800 | RPS19 | Pf3D7_13_v3: 739,823 . . . 741,699(+) |
| PF3D7_1203700 | NAPL | Pf3D7_12_v3: 185,860 . . . 188,842(−) |
| PF3D7_1424100 | RPL5 | Pf3D7_14_v3: 973,577 . . . 977,414(−) |
| PF3D7_0903900 | RPL32 | Pf3D7_09_v3: 184,821 . . . 186,458(+) |
| PF3D7_1471100 | EXP2 | Pf3D7_14_v3: 2,903,947 . . . 2,907,414(−) |
| PF3D7_0925900 | LCN | Pf3D7_09_v3: 1,039,462 . . . 1,042,118(+) |
| PF3D7_0913200 | EF-1beta | Pf3D7_09_v3: 574,522 . . . 576,690(+) |
| PF3D7_1447000 | N/A | Pf3D7_14_v3: 1,928,504 . . . 1,931,646(+) |
| PF3D7_1408600 | N/A | Pf3D7_14_v3: 322,243 . . . 324,322(+) |
| PF3D7_0322000 | CYP19A | Pf3D7_03_v3: 928,124 . . . 929,640(−) |
| PF3D7_0617800 | H2A | Pf3D7_06_v3: 747,329. . . 749,285(−) |
| PF3D7_0320900 | H2A.Z | Pf3D7_03_v3: 874,313 . . . 876,685(+) |
| PF3D7_1033100 | AdoMetDC/ODC | Pf3D7_10_v3: 1,323,813 . . . 1,331,955(−) |
| PF3D7_1431700 | N/A | Pf3D7_14_v3: 1,246,677 . . . 1,248,407(+) |
| PF3D7_1330800 | N/A | Pf3D7_13_v3: 1,293,638 . . . 1,297,395(+) |
| PF3D7_1130100 | RPL38 | Pf3D7_11_v3: 1,162,731 . . . 1,164,039(−) |
| PF3D7_1457200 | TRX1 | Pf3D7_14_v3: 2,343,508 . . . 2,344,657(−) |
| PF3D7_1451100 | eEF2 | Pf3D7_14_v3: 2,089,730 . . . 2,094,221(+) |
| PF3D7_0522000 | N/A | Pf3D7_05_v3: 895,431 . . . 897,323(+) |
| PF3D7_1027800 | RPL3 | Pf3D7_10_v3: 1,153,081 . . . 1,155,488(−) |
| PF3D7_1224300 | PABP1 | Pf3D7_12_v3: 985,545 . . . 993,212(+) |
| PF3D7_1426000 | RPL21 | Pf3D7_14_v3: 1,015,470 . . . 1,017,199(−) |
| PF3D7_0415900 | N/A | Pf3D7_04_v3: 697,475 . . . 700,443(−) |
| PF3D7_0511000 | TCTP | Pf3D7_05_v3: 467,139 . . . 468,451(−) |
| PF3D7_1424400 | N/A | Pf3D7_14_v3: 976,472 . . . 980,866(+) |
| PF3D7_0716300 | N/A | Pf3D7_07_v3: 716,275 . . . 719,340(+) |
| PF3D7_1130200 | PfP0 | Pf3D7_11_v3: 1,163,768 . . . 1,167,334(+) |
| PF3D7_0312800 | N/A | Pf3D7_03_v3: 533,606 . . . 535,480(+) |
| PF3D7_1309100 | N/A | Pf3D7_13_v3: 417,668 . . . 420,588(+) |
| PF3D7_0610400 | H3 | Pf3D7_06_v3: 444,363 . . . 445,584(+) |
| PF3D7_0818200 | 14-3-3I | Pf3D7_08_v3: 827,788 . . . 832,382(+) |
| PF3D7_0422400 | RPS19 | Pf3D7_04_v3: 1,025,510 . . . 1,027,735(+) |
| PF3D7_1212700 | EIF3A | Pf3D7_12_v3: 557,007 . . . 564,177(−) |
| PF3D7_1004000 | N/A | Pf3D7_10_v3: 179,019 . . . 180,699(−) |
| PF3D7_1248700 | N/A | Pf3D7_12_v3: 1,993,096 . . . 2,001,002(+) |
| PF3D7_1120000 | N/A | Pf3D7_11_v3: 755,951 . . . 760,604(+) |
| PF3D7_1242700 | N/A | Pf3D7_12_v3: 1,803,130 . . . 1,804,735(+) |
| PF3D7_1342600 | MyoA | Pf3D7_13_v3: 1,673,239 . . . 1,677,299(+) |
| PF3D7_0307200 | N/A | Pf3D7_03_v3: 314,714 . . . 316,835(−) |
| PF3D7_1460300 | RPL29 | Pf3D7_14_v3: 2,459,048 . . . 2,460,369(−) |
| PF3D7_1026000 | N/A | Pf3D7_10_v3: 1,098,008 . . . 1,100,175(−) |
| PF3D7_0207600 | SERA5 | Pf3D7_02_v3: 303,409 . . . 307,376(−) |
| PF3D7_1465900 | N/A | Pf3D7_14_v3: 2,683,497 . . . 2,685,654(+) |
| PF3D7_1356900 | PK5 | Pf3D7_13_v3: 2,262,738 . . . 2,265,705(−) |
| PF3D7_0418300 | N/A | Pf3D7_04_v3: 819,175 . . . 826,740(−) |
| PF3D7_0708800 | HSP110c | Pf3D7_07_v3: 393,813 . . . 399,112(+) |
| PF3D7_1460700 | RPL27 | Pf3D7_14_v3: 2,475,440 . . . 2,476,914(−) |
| PF3D7_1029600 | ADA | Pf3D7_10_v3: 1,204,815 . . . 1,207,912(+) |
| PF3D7_1414300 | N/A | Pf3D7_14_v3: 570,027 . . . 571,644(+) |
| PF3D7_1211800 | PfpUB | Pf3D7_12_v3: 525,310 . . . 528,190(+) |
| PF3D7_1022400 | SRSF4 | Pf3D7_10_v3: 939,330 . . . 942,546(−) |
| PF3D7_0730800 | N/A | Pf3D7_07_v3: 1,320,155 . . . 1,323,192(+) |
| PF3D7_1223100 | PKAr | Pf3D7_12_v3: 927,063 . . . 930,026(−) |
| PF3D7_0628300 | CEPT | Pf3D7_06_v3: 1,169,270 . . . 1,174,371(−) |
| PF3D7_1200600 | VAR2CSA | Pf3D7_12_v3: 45,887 . . . 58,295(−) |
| PF3D7_0826700 | RACK1 | Pf3D7_08_v3: 1,159,770 . . . 1,162,786(−) |
| PF3D7_1003600 | IMC1c | Pf3D7_10_v3: 164,213 . . . 166,726(+) |
| PF3D7_0817900 | HMGB2 | Pf3D7_08_v3: 816,742 . . . 818,583(−) |
| PF3D7_1308300 | RPS27 | Pf3D7_13_v3: 370,778 . . . 373,004(+) |
| PF3D7_0935900 | REX1 | Pf3D7_09_v3: 1,419,960 . . . 1,424,840(−) |
| PF3D7_0406200 | Pfs16 | Pf3D7_04_v3: 328,473 . . . 330,639(+) |
| PF3D7_1033400 | HAD1 | Pf3D7_10_v3: 1,339,140 . . . 1,341,120(+) |
| PF3D7_1105400 | RPS4 | Pf3D7_11_v3: 235,266 . . . 238,856(−) |
| PF3D7_1026800 | RPS2 | Pf3D7_10_v3: 1,121,930 . . . 1,124,959(+) |
| PF3D7_0503400 | ADF1 | Pf3D7_05_v3: 139,481 . . . 142,206(+) |
| PF3D7_0730300 | ApiAP2 | Pf3D7_07_v3: 1,297,144 . . . 1,302,090(+) |
| PF3D7_0519400 | RPS24 | Pf3D7_05_v3: 797,479 . . . 799,233(−) |
| PF3D7_1006800 | GBP2 | Pf3D7_10_v3: 276,414 . . . 280,198(+) |
| PF3D7_1001500 | ETRAMP10 | Pf3D7_10_v3: 80,302 . . . 82,576(−) |
| PF3D7_0611700 | RPL39 | Pf3D7_06_v3: 488,418 . . . 489,504(−) |
| PF3D7_0322900 | RPS3A | Pf3D7_03_v3: 965,293 . . . 967,150(−) |
| PF3D7_0922200 | SAMS | Pf3D7_09_v3: 901,584 . . . 905,953(+) |
| PF3D7_1129100 | PV1 | Pf3D7_11_v3: 1,124,682 . . . 1,128,048(+) |
| PF3D7_0423700 | ETRAMP4 | Pf3D7_04_v3: 1,069,829 . . . 1,072,039(−) |
| PF3D7_1019400 | N/A | Pf3D7_10_v3: 785,374 . . . 787,414(+) |
| PF3D7_1120100 | PGM1 | Pf3D7_11_v3: 760,859 . . . 763,812(+) |
| PF3D7_1142600 | N/A | Pf3D7_11_v3: 1,707,861 . . . 1,709,391(−) |
| PF3D7_1108600 | ERC | Pf3D7_11_v3: 369,068 . . . 371,707(−) |
| PF3D7_0317600 | RPS11 | Pf3D7_03_v3: 724,394 . . . 726,450(+) |
| PF3D7_0520000 | RPS9 | Pf3D7_05_v3: 828,277 . . . 830,137(+) |
| PF3D7_1341200 | N/A | Pf3D7_13_v3: 1,633,454 . . . 1,635,827(−) |
| PF3D7_1323400 | RPL23 | Pf3D7_13_v3: 971,388 . . . 973,888(−) |
| PF3D7_0217800 | RPS26 | Pf3D7_02_v3: 733,477 . . . 734,855(+) |
| PF3D7_0307100 | RPS12 | Pf3D7_03_v3: 312,534 . . . 313,923(−) |
| PF3D7_0831700 | HSP70x | Pf3D7_08_v3: 1,364,640 . . . 1,369,862(−) |
| PF3D7_1351400 | N/A | Pf3D7_13_v3: 2,051,487 . . . 2,053,580(+) |
| PF3D7_0501300 | SBP1 | Pf3D7_05_v3: 67,224 . . . 70,944(+) |
| PF3D7_1441100 | N/A | Pf3D7_14_v3: 1,673,090 . . . 1,677,720(−) |
| PF3D7_1342000 | N/A | Pf3D7_13_v3: 1,652,531 . . . 1,655,349(−) |
| PF3D7_0814000 | N/A | Pf3D7_08_v3: 679,355 . . . 681,960(+) |
| PF3D7_0507100 | RPL4 | Pf3D7_05_v3: 294,476 . . . 297,505(−) |
| PF3D7_0922500 | PGK | Pf3D7_09_v3: 912,487 . . . 915,584(+) |
| PF3D7_1035200 | N/A | Pf3D7_10_v3: 1,393,856 . . . 1,397,323(+) |
| PF3D7_1229400 | MIF | Pf3D7_12_v3: 1,208,198 . . . 1,209,870(+) |
| PF3D7_0607000 | N/A | Pf3D7_06_v3: 291,965 . . . 297,805(+) |
| PF3D7_1251200 | N/A | Pf3D7_12_v3: 2,091,731 . . . 2,094,720(+) |
| PF3D7_1401400 | ETRAMP14 | Pf3D7_14_v3: 52,256 . . . 54,457(−) |
| PF3D7_0520900 | SAHH | Pf3D7_05_v3: 856,500 . . . 859,367(+) |
| PF3D7_0721100 | N/A | Pf3D7_07_v3: 915,483 . . . 917,854(+) |

*Gene Identifier (ID) according to PlasmoDB (https://plasmodb.org/plasmo/app/) of the Eukaryotic Pathogen, Vector and Host Informatics Resource (VEuPathDB) of the US National Institute of Allergy and Infectious Diseases.

In some embodiments, the heterologous nucleic acid molecule is site specifically integrated into a gene locus set forth in Table 2. In some embodiments, a plurality of copies of the heterologous nucleic acid molecule are site specifically integrated into a single gene locus set forth in Table 2. In some embodiments, the heterologous nucleic acid molecule is site specifically integrated into at least one gene locus set forth in Table 2 and at least one second different gene locus. In some embodiments, the heterologous nucleic acid molecule is site specifically integrated into at least two different gene loci set forth in Table 2. In some embodiments, the heterologous nucleic acid molecule is site specifically integrated into at least one gene locus set forth in Table 2 and a second different gene locus that is not set forth in Table 2.

In some embodiments, the heterologous nucleic acid molecule is site specifically integrated into one or more of the following gene loci GAPDH, HSP70, MESA, MSP9, HGPRT, ENO, HSP90, PMT, H2B, LDH, FBPA, GBP130, ACT1, CK1, PyrK, NT1, EBA175, ETRAMP2, ALBA1, BIP, nPrx, RPL2, MSP1, RESA, RESA3, ETRAMP11.2, eIF4A, ETRAMP5, H2B.Z, PREBP, RPP1, PDI8, EXP1, RhopH3, RAN, P23, PNP, NAPS, IMC1g, RPS19, NAPL, RPL5, RPL32, EXP2, LCN, EF-1beta, CYP19A, H2A, H2A.Z, AdoMetDC/ODC, RPL38, TRX1, eEF2, RPL3, PABP1, RPL21, TCTP, PfP0, H3, 14-3-3I, RPS19, EIF3A, MyoA, RPL29, SERA5, PK5, HSP110c, RPL27, ADA, PfpUB, SRSF4, PKAr, CEPT, VAR2CSA, RACK1, IMC1c, HMGB2, RPS27, REX1, Pfs16, HAD1, RPS4, RPS2, ADF1, ApiAP2, RPS24, GBP2, ETRAMP10, RPL39, RPS3A, SAMS, PV1, ETRAMP4, PGM1, ERC, RPS11, RPS9, RPL23, RPS26, RPS12, HSP70x, SBP1, RPL4, PGK, MIF, ETRAMP14, or SAHH.

In some embodiments, more than one copy of the heterologous nucleic acid molecule is site specifically integrated into one or more of the following gene loci GAPDH, HSP70, MESA, MSP9, HGPRT, ENO, HSP90, PMT, H2B, LDH, FBPA, GBP130, ACT1, CK1, PyrK, NT1, EBA175, ETRAMP2, ALBA1, BIP, nPrx, RPL2, MSP1, RESA, RESA3, ETRAMP11.2, eIF4A, ETRAMP5, H2B.Z, PREBP, RPP1, PDI8, EXP1, RhopH3, RAN, P23, PNP, NAPS, IMC1g, RPS19, NAPL, RPL5, RPL32, EXP2, LCN, EF-1beta, CYP19A, H2A, H2A.Z, AdoMetDC/ODC, RPL38, TRX1, eEF2, RPL3, PABP1, RPL21, TCTP, PfP0, H3, 14-3-3I, RPS19, EIF3A, MyoA, RPL29, SERA5, PK5, HSP110c, RPL27, ADA, PfpUB, SRSF4, PKAr, CEPT, VAR2CSA, RACK1, IMC1c, HMGB2, RPS27, REX1, Pfs16, HAD1, RPS4, RPS2, ADF1, ApiAP2, RPS24, GBP2, ETRAMP10, RPL39, RPS3A, SAMS, PV1, ETRAMP4, PGM1, ERC, RPS11, RPS9, RPL23, RPS26, RPS12, HSP70x, SBP1, RPL4, PGK, MIF, ETRAMP14, or SAHH.

In some embodiments, one or more copy of the heterologous nucleic acid molecule is site specifically integrated into one or more of the following gene loci GAPDH, HSP70, MESA, MSP9, HGPRT, ENO, HSP90, PMT, H2B, LDH, FBPA, GBP130, ACT1, CK1, PyrK, NT1, EBA175, ETRAMP2, ALBA1, BIP, nPrx, RPL2, MSP1, RESA, RESA3, ETRAMP11.2, eIF4A, ETRAMP5, H2B.Z, PREBP, RPP1, PDI8, EXP1, RhopH3, RAN, P23, PNP, NAPS, IMC1g, RPS19, NAPL, RPL5, RPL32, EXP2, LCN, EF-1beta, CYP19A, H2A, H2A.Z, AdoMetDC/ODC, RPL38, TRX1, eEF2, RPL3, PABP1, RPL21, TCTP, PfP0, H3, 14-3-3I, RPS19, EIF3A, MyoA, RPL29, SERA5, PK5, HSP110c, RPL27, ADA, PfpUB, SRSF4, PKAr, CEPT, VAR2CSA, RACK1, IMC1c, HMGB2, RPS27, REX1, Pfs16, HAD1, RPS4, RPS2, ADF1, ApiAP2, RPS24, GBP2, ETRAMP10, RPL39, RPS3A, SAMS, PV1, ETRAMP4, PGM1, ERC, RPS11, RPS9, RPL23, RPS26, RPS12, HSP70x, SBP1, RPL4, PGK, MIF, ETRAMP14, or SAHH; and one or more copy of the heterologous nucleic acid molecule is site-specifically integrated into one or more gene loci not set forth above.

Multiple copies of the heterologous nucleic acid molecule can be integrated into the target genome, e.g., multiple copies can be integrated into the same gene locus; or one or more copies can be integrated into multiple different gene loci. In some embodiments, multiple copies of the heterologous nucleic acid molecule are integrated into a single gene locus. In some embodiments, multiple copies of the heterologous nucleic acid molecule are integrated into a target genome into at least two different genetic loci.

In some embodiments, a recombinase landing pad system can be utilized for integration of the heterologous nucleic acid molecule into a specific locus. Recombinase landing pad systems are known in the art, see, e.g., Gaidukov, Leonid et al. “A multi-landing pad DNA integration platform for mammalian cell engineering.” Nucleic acids research vol. 46, 8 (2018): 4072-4086. doi:10.1093/nar/gky216, the entire contents of which are incorporated by reference herein for all purposes. Generally, a landing pad containing a recombinase recognition site (e.g., an attP site (e.g., a Bxb1 attP)) and one or more selectable marker is integrated into the target locus of the genome. The corresponding recombinase (e.g., a serine integrase (e.g., Bxb1)) can encoded in the landing pad or introduced separately (e.g., introduced using a separate plasmid encoding the recombinase). A nucleic acid molecule encoding the heterologous nucleic acid molecule and a corresponding recombinase recognition site (e.g., an attB site (e.g., a Bxb1 attB)) is introduced, wherein the recombinase mediates the site-specific integration of the heterologous nucleic acid.

### 5.3.1 Genetic Engineering Systems

Any suitable genetic engineering system known in the art may be employed to effectuate the insertion of a heterologous nucleic acid molecule (e.g., described herein) encoding the therapeutic protein (e.g., described herein) into a genome of the Plasmodium. Standard genetic engineering systems known in the art include, but are not limited to, CRISPR/Cas systems, transcription activator-like effector nuclease (TALEN) systems, zinc finger nuclease (ZNF) systems, and meganuclease systems. Additional suitable genetic engineering systems known in the art can be used, see, e.g., Ribeiro J. et al. Guide RNA selection for CRISPR-Cas9 transfections in Plasmodium falciparum, International Journal for Parasitology, 48(11): 825-832 (2018) 825-832, https://doi.org/10.1016/j.ijpara.2018.03.009 (hereinafter “Ribeiro 2018”); Silva, George et al. “Meganucleases and other tools for targeted genome engineering: perspectives and challenges for gene therapy.” Current gene therapy vol. 11, 1 (2011): 11-27. doi:10.2174/156652311794520111 (hereinafter “Silva 2011”), the full contents of each of which is incorporated by reference herein for all purposes.

### 5.3.1.1 Engineered CRISPR/Cas Systems

CRISPR/Cas genetic engineering systems are known in the art and comprise (i) one or more RNA-guided endonuclease (e.g., a Cas protein) (e.g., a nickase) to cleave the genomic DNA and (ii) one or more gRNA (e.g., a sgRNA) to bring the RNA-guided endonuclease to the target region of the genomic DNA. General information on CRISPR/Cas systems, components thereof, testing, and delivery of such components, including e.g., methods, materials, delivery vehicles, vectors, particles, AAV, and methods of making and using thereof, including as to amounts and formulations can be found in the art, see, e.g., Makarova et al. (2018) The CRISPR Journal 1(5): 325-336; and Adli (2018) Nat. Communications 9: 1911, Silva 2011, Makarova K S, Koonin E V. Annotation and Classification of CRISPR-Cas Systems. Methods Mol Biol. 2015; 1311:47-75. doi:10.1007/978-1-4939-2687-9_4; WO2014093595A1, WO2014093622A2, WO2014093635A1, WO2014093655A2, WO2014093661A2, WO2014093694A1, WO2014093701A1, WO2014093709A1, WO2014093712A1, WO2014093718A1, WO2014204723A1, WO2014204724A1, WO2014204725A1, WO2014204726A1, WO2014204727A1, WO2014204728A1, WO2014204729A1, WO2020047124A1, WO2021178709A1, WO2021178717A2, WO2021178720A2, WO2021248102A1, the entire contents of each of which is incorporated by reference herein in their entirety for all purposes.

In some embodiments, the endonuclease is a nickase. In some embodiments, the endonuclease is modified (e.g., the amino acid sequence of the endonuclease is modified) to have nickase activity.

In some embodiments, the gRNA comprises one or more of a sgRNA, a tracrRNA, or a sgRNA. It is known in the art that certain RNA-guided endonucleases require both a sgRNA and a tracrRNA; while other RNA-guided endonucleases require only e.g., a crRNA (e.g., CasΦ). See, e.g., Pausch, Patrick et al. “CRISPR-CasΦ from huge phages is a hypercompact genome editor.” Science (New York, N.Y.) vol. 369, 6501 (2020): 333-337. doi:10.1126/science.abb1400, the entire contents of which are incorporated herein by reference for all purposes.

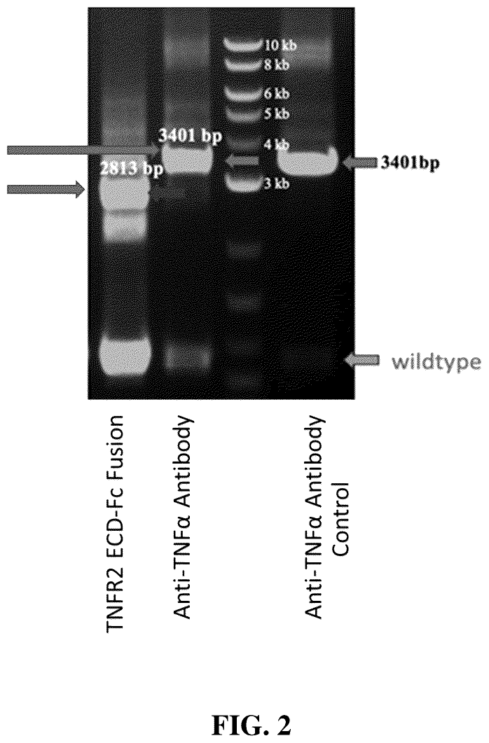

In some embodiments, the gRNA of the system is a sgRNA. In some embodiments, the gRNA of the system is a tracrRNA. In some embodiments, the gRNA of the system is a tracrRNA in combination with a crRNA. In some embodiments, the gRNA is a sgRNA. The crRNA or the crRNA portion of a sgRNA can be designed to target a specific genomic DNA sequence by a person of ordinary skill in the art using standard methods known in the art. For example, by designing the crRNA to be substantially complementary to the target genomic DNA. Likewise, the tracrRNA or the tracrRNA portion of a sgRNA can be designed recognize a specific RNA-guided endonuclease (e.g., a specific Cas protein) by standard methods known in the art. The nucleotide sequences of tRNAs that recognize specific RNA-guided endonucleases are also known in the art, see, e.g., Graf R, et al. sgRNA Sequence Motifs Blocking Efficient CRISPR/Cas9-Mediated Gene Editing. Cell Rep. 2019; 26(5):1098-1103.e3. doi:10.1016/j.celrep.2019.01.024 (e.g., supplemental FIG. 2A), the full contents of which are incorporated by reference herein.

In some embodiments, the RNA-guided endonuclease is a Cas protein (or a functional fragment or variant thereof). In some embodiments, the Cas protein is a Cas9 protein (or a functional fragment or variant thereof). In some embodiments, the Cas9 protein is derived from S. pyogenes, S. thermophiles, N. meningitidis, or C. jejuni. In some embodiments, the Cas9 protein is derived from S. pyogenes or S. thermophiles. Suitable RNA-guided endonucleases include, without limitation, Class I and Class II CRISPR-associated endonucleases. Class I is divided into types I, III, and IV, and includes, without limitation, type I (Cas3), type LA (Cas8a, Cas5), type LB (Cas8b), type LC (Cas8c), type LD (Cas10d), type LE (Cse1, Cse2), type LF (Csy1, Csy2, Csy3), type LU (GSU0054), type III (Cas10), type IILA (Csm2), type IILB (Cmr5), type IILC (Csx10 or Csx11), type IILD (Csx10), and type IV (Csf1). Class II is divided into types II, V, and VI, and includes, without limitation, type II (Cas9), type ILA (Csn2), type ILB (Cas4), type V (Cpf1, C2c1, C2c3), and type VI (Cas13a, Cas13b, Cas13c). RNA-guided endonucleases also include naturally occurring Class II CRISPR endonucleases such as Cas9 (Type II) or Cas12a/Cpf1 (Type V), as well as other nucleases derived or obtained therefrom. In some embodiments, the Cas9 protein is S. pyogenes Cas9 (SpCas9), S. aureus Cas9 (SaCas9), N. meningitidis Cas9 (NmCas9), C. jejuni Cas9 (CjCas9), Geobacillus Cas9 (GeoCas9), S. pyogenes Cas9 D10A nickase (SpCas9n), S. aureus Cas9 high fidelity R691A (Cas9-HF), S. pyogenes Cas9-H840A (SpCas9-H840A), FokI-dCas9 (catalytically inactive Cas9) (or a functional fragment or variant of any of the foregoing).

### 5.3.1.2 Engineered TALENs

A TALEN genetic engineering system refers to a system that employs (i) one or more TALE-DNA binding domain and (ii) one or more endonuclease domain, such as FokI cleavage domain. The TALE-DNA-binding domain(s) comprises one or more TALE repeat unit, each having about 30-40 amino acids. In some embodiments, the one or more TALE-DNA-binding domain is operably connected to the FokI cleavage domain (e.g., directly or via a linker). The TALEN-DNA-binding domain can be designed to target a specific genomic DNA sequence by a person of ordinary skill in the art using standard methods known in the art, e.g., by designing the nucleotide TALE-DNA binding domain to be substantially complementary to the target genomic sequence. General information on TALEN genetic engineering systems, components thereof, testing, and delivery of such components, including e.g., methods, materials, delivery vehicles, vectors, particles, and methods of making and using can be found in the art, see, e.g., Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29: 143-148; Zhang et al. (2011) Nat. Biotechnol. 29: 149-153; and Reyon et al. (2012) Nat. Biotechnol. 30(5): 460-465, Silva 2011, the full contents of each of which is incorporated by reference herein in their entirety for all purposes.

### 5.3.1.3 Engineered ZFNs

ZFN genetic engineering systems refer to systems comprising one or more ZFN that comprises (i) one or more ZNF-DNA-binding domain and (ii) one or more DNA-cleavage domain, such as FokI cleavage domain. In some embodiments, the one or more ZNF-DNA-binding domain is operably connected to the DNA-cleavage domain (e.g., a FokI cleavage domain) (e.g., directly or via a linker). The ZNF-DNA-binding domain can be designed to target a specific genomic DNA sequence by a person of ordinary skill in the art using standard methods known in the art e.g., by designing the nucleotide ZNF-DNA binding domain to be substantially complementary to the target genomic sequence. General information on ZFN genetic engineering systems, components thereof, and delivery of such components, including e.g., methods, materials, delivery vehicles, vectors, particles, and methods of making and using can be found in the art, see, e.g., Porteus and Baltimore (2003) Science 300: 763; Miller et al. (2007) Nat. Biotechnol. 25:778-785; Sander et al. (2011) Nature Methods 8:67-69; and Wood et al. (2011) Science 333:307, Silva 2011, the full contents of each of which is incorporated by reference herein in their entirety for all purposes.

### 5.3.1.4 Engineered Meganucleases

Systems comprising an engineered meganuclease may also be suitable as a genetic engineered system for use in the methods described herein. Meganucleases contain large DNA-binding domains that can be engineered to target a specific genomic DNA sequence by a person of ordinary skill in the art using standard methods known in the art, e.g., by designing the DNA binding domain of the meganuclease to be substantially complementary to the target genomic sequence. General information on meganuclease genetic engineering systems, components thereof, testing, and delivery of such components, including e.g., methods, materials, delivery vehicles, vectors, particles, and methods of making and using can be found in the art, see, e.g., Silva 2011, the full contents of each of which is incorporated by reference herein in their entirety for all purposes.

### 5.4 Scalable Manufacturing of Engineered Plasmodia

Also provided herein are compositions and methods of large-scale manufacturing of engineered Plasmodia described herein. In one aspect, provided herein are bioreactors (e.g., 500L, 1000L, 2000L) comprising a population of engineered Plasmodia described herein. The bioreactor may include any suitable culture medium (e.g., as described herein). See, e.g., Talman 2010 (describing the culture of P. falciparum 3D7 in 4% haematocrit RPMI 1640 (Gibco, UK), supplemented with 10% human AB serum, and gassed with 5% CO2, 0.5% O2 in N2 at 37° C.); and Hoppe 1991) (describing the culture of P. falciparum 3D7 parasites in a complete medium at 1% haematocrit at 37° C. in a 5% CO₂/3% O₂/balanced N₂ gas mixture), and Schuster 2002.

In some embodiments, the culture medium contains hRBCs. In some embodiments, fresh hRBCs are added to the bioreactor at set intervals (e.g., fed-batch culture) or continuously (e.g., continuous culture). Additional parameters of the bioreactor (e.g., parasitemia, hematocrit, load, temperature, culture mode (e.g., batch, fed-batch, continuous, dissolved oxygen, pH, etc.) can be determined by a person of ordinary skill in the art using routine experimentation (e.g., as described herein).

In some embodiments, the engineered Plasmodia are (i) isolated from the bioreactor and any non-Plasmodia components (e.g., culture media, RBCs) (e.g., as described herein), (ii) purified from any non-Plasmodia components (e.g., culture media, RBCs) (e.g., as described herein), (iii) formulated into a pharmaceutical composition (e.g., a pharmaceutical composition described herein), (iv) assessed for viability (e.g., as described herein), (v) assessed for purity using one or more assays (e.g., standard assays known in the art, e.g., assays for substantial absence of endotoxin, viral contamination, antibiotics, and other process impurities such as column materials), (vi) assessed for expression, protein activity and/or potency, (vii) distributed into a suitable container, (viii) packaged (e.g., into a kit described herein), (ix) stored at a suitable temperature (e.g., −20° C., −4° C.), and/or shipped, or any combination thereof.

In some embodiments, the bioreactor can accommodate a volume of at least about 500 L, 1000 L, 2000 L, 3000 L, 4000 L, 5000 L, 6000 L, 7000 L, 8000 L, 9000 L, or 10000 L.

### 5.5 Methods of Making Therapeutic Proteins In Vitro Using Plasmodia

Provided herein are various methods of making therapeutic proteins (e.g., a therapeutic protein described herein) utilizing an engineered Plasmodium described herein. In one aspect, provided herein are methods of making a therapeutic protein (e.g., a therapeutic protein described herein) (e.g., in vitro, ex vivo) utilizing an engineered Plasmodium described herein.

In some embodiments, provided herein are methods of making a therapeutic protein (e.g., a therapeutic protein described herein), the method comprising: culturing an engineered Plasmodium (or a population of engineered Plasmodia) described herein in a composition comprising hRBCs, under conditions and for a period of time sufficient for (i) infection of the hRBCs by the engineered Plasmodium and (ii) expression the therapeutic protein, to thereby make a therapeutic protein.

In some embodiments, provided herein are methods of making a therapeutic protein, the method comprising culturing hRBCs comprising an engineered Plasmodium described herein in the presence of a population of hRBCs under conditions and for a period of time sufficient for expression of the therapeutic protein, to thereby make a therapeutic protein.

In some embodiments, the hRBCs comprising the engineered Plasmodium are cultured in the presence of the population of RBCs under conditions and for a period of time sufficient for (i) release of the engineered Plasmodium from the hRBCs, (ii) infection of the population of hRBCs by the engineered Plasmodium, and (iii) expression of the therapeutic protein.

In some embodiments, the therapeutic protein is secreted from the hRBCs. In some embodiments, the therapeutic protein is not secreted from the hRBCs. In some embodiments, the culturing results in the rupture of hRBCs infected with the engineered Plasmodium. In some embodiments, the rupture results in the release of the therapeutic protein.

In some embodiments, the composition comprising hRBCs is whole human blood or a composition comprising isolated hRBCs. In some embodiments, the composition comprising hRBCs is whole human blood. In some embodiments, the method comprises isolating the serum of the whole human blood. In some embodiments, the serum comprises the therapeutic protein.

In some embodiments, the method further comprises synchronizing the population of engineered Plasmodia to a single developmental stage. Standard methods of synchronization are known in the art and include for example Percoll/sorbitol synchronization and Percoll density centrifugation; see, e.g., Hoppe 1991 and Childs 2013.

In some embodiments, the composition comprising the hRBCs is whole human blood (e.g., a whole human blood sample, e.g., obtained from a subject). In some embodiments, the composition comprising the hRBCs comprises isolated hRBCs (e.g., hRBCs isolated from a whole blood sample e.g., obtained from a subject). Whole human blood samples can be obtained from a human subject using standard methods known in the art, e.g., venous blood draw procedures. hRBCs can be isolated from a sample of whole blood using standard methods known in the art, including, e.g., density gradient centrifugation.

The engineered Plasmodium (or population of engineered Plasmodia) can be cultured in any suitable culture medium. Standard culture mediums for in vitro culture of Plasmodia are known in the art, see, e.g., Talman 2010 (describing the culture of P. falciparum 3D7 in 4% haematocrit RPMI 1640 (Gibco, UK), supplemented with 10% human AB serum, and gassed with 5% CO2, 0.5% O2 in N2 at 37° C.); and Hoppe 1991) (describing the culture of P. falciparum 3D7 parasites in a complete medium at 1% haematocrit at 37° C. in a 5% CO₂/3% O₂/balanced N₂ gas mixture); and Schuster 2002.

In some embodiments, the therapeutic protein is secreted from the hRBCs. In these instances the therapeutic protein can be isolated directly from the culture medium. If the therapeutic protein is expressed intracellularly, the therapeutic protein can be released from the hRBCs into the culture medium using standard methods known in the art, e.g., hRBC lysis and purification. See, e.g., Mutungi 2015; Radfar 2009. In some embodiments, the method comprises isolating the therapeutic protein. In some embodiments, the method comprises purifying the therapeutic protein. The produced therapeutic protein may be isolated from the Plasmodium cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The therapeutic protein may be concentrated, buffer exchanged by ultrafiltration, and the retentate from the ultrafiltration may be filtered through an appropriate filter, e.g., a 0.22 μm filter. See, e.g., U.S. Pat. No. 5,762,939, the entire contents of which is incorporated by reference herein for all purposes.

In some embodiments, the method comprises measuring the level or concentration of therapeutic protein produced. The level or concentration of the therapeutic protein can be measured according to standard methods known in the art. See, e.g., Kielkopf C L, Bauer W, Urbatsch I L. Methods for Measuring the Concentrations of Proteins. Cold Spring Harb Protoc. 2020; 2020(4):102277. Published 2020 Apr. 1. doi:10.1101/pdb.top102277; and Noble J E, Bailey M J. Quantitation of protein. Methods Enzymol. 2009; 463:73-95. doi:10.1016/S0076-6879(09)63008-1; the entire contents of each of which are incorporated herein by reference for all purposes.

Appropriate assays can be determined by a person of ordinary skill in the art to determine, e.g., a functional characteristic of a specific protein. For example, binding affinity of a therapeutic protein produced by the methods described herein for another protein can be determined using standard methods known in the art. For example, binding affinity can be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

### 5.6 Pharmaceutical Compositions

In one aspect, provided herein are pharmaceutical compositions comprising an engineered Plasmodium (or population of engineered Plasmodia) described herein or a therapeutic protein made by a method described herein (see, e.g., § 5.5), and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing an engineered Plasmodium (or population of engineered Plasmodia) described herein or a therapeutic protein made by a method described herein and formulating it into a pharmaceutically acceptable composition by the addition of one or more pharmaceutically acceptable excipient.

In some embodiments, the pharmaceutical composition comprises a therapeutic protein made by a method described herein (see, e.g., § 5.5), and a pharmaceutically acceptable excipient.

In some embodiments, the pharmaceutical composition comprises serum. In some embodiments, the serum is from a subject. In some embodiments, the serum is from a subject in need of treatment with the therapeutic protein.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

A pharmaceutical composition may be formulated for any route of administration to a subject. The skilled person knows the various possibilities to administer a pharmaceutical composition described herein. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.7 Methods of Use

Provided herein are various methods of utilizing the engineered Plasmodium (or population of engineered Plasmodia) described herein, the therapeutic proteins made by the methods described herein (see, e.g., § 5.5), and pharmaceutical compositions described herein. Exemplary subjects include mammals, e.g., humans, non-human mammals, e.g., non-human primates. In some embodiments, the subject is a human.

In some embodiments, the engineered Plasmodium (or population of engineered Plasmodia) described herein can be used to deliver a protein to a tissue or organ ex-vivo. In embodiments, the engineered Plasmodium (or population of engineered Plasmodia) described herein can be used to deliver a protein to a subject in vivo.

In embodiments, the engineered Plasmodium (or population of engineered Plasmodia) described herein can be used to produce a protein in vitro and the produced protein can be delivered to a tissue or organ ex vivo or to a subject in vivo.

The dosage of an engineered Plasmodium (or population of engineered Plasmodia) described herein, a therapeutic protein made by a method described herein, or a pharmaceutical composition described herein to be administered to a subject in accordance with any of the methods described herein can be determined in accordance with standard techniques well known to those of ordinary skill in the art, including the route of administration, the age and weight of the subject, and the type (if any) adjuvant is used.

In some embodiments, the engineered Plasmodium (or population of engineered Plasmodia) is administered to the subject, and the subject does not exhibit or only exhibits a mild symptomatic immune response to the engineered Plasmodium or (or population of engineered Plasmodia).

### 5.7.1 Delivery

In one aspect, provided herein are methods of delivering an engineered Plasmodium (or population of engineered Plasmodia) described herein, a therapeutic protein made by a method described herein, or a pharmaceutical composition described herein to a subject, the method comprising administering to the engineered Plasmodium (or population of engineered Plasmodia), the therapeutic protein made by a method described herein, or the pharmaceutical composition to the subject, to thereby deliver the engineered Plasmodium (or population of engineered Plasmodia), the therapeutic protein made by a method described herein, or the pharmaceutical composition to the subject. In some embodiments, the engineered Plasmodium (or population of engineered Plasmodia), the therapeutic protein made by a method described herein, or the pharmaceutical composition is administered to the subject in an amount and for a time sufficient to deliver the engineered Plasmodium (or population of engineered Plasmodia), the therapeutic protein made by a method described herein, or the pharmaceutical composition to the subject.

In some embodiments, the therapeutic protein made by a method described herein is administered to the subject, wherein the hRBCs utilized in the method of making the therapeutic protein, are autologous to the subject. In some embodiments, the therapeutic protein made by a method described herein is administered to the subject, wherein the hRBCs utilized in the method of making the therapeutic protein, are allogenic to the subject.

### 5.7.2 Treatment, Amelioration, and Prevention of Diseases

In one aspect, provided herein are methods of treating, ameliorating, or preventing a disease in a subject in need thereof, the method comprising administering an engineered Plasmodium (or population of engineered Plasmodia) described herein, a therapeutic protein made by a method described herein, or a pharmaceutical composition described herein to the subject, to thereby treat the disease in the subject. These methods of treating, ameliorating, or preventing a disease in the context of the present disclosure are used interchangeably with the engineered Plasmodium, the therapeutic protein made by a method described herein, or the pharmaceutical composition described herein for use in such methods. In some embodiments, the engineered Plasmodium (or population of engineered Plasmodia) described herein, the therapeutic protein made by a method described herein, or the pharmaceutical composition described herein is administered to the subject in an amount and for a time sufficient to treat, ameliorate, or prevent the disease in the subject. Further disclosed is an engineered Plasmodium (or population of engineered Plasmodia) described herein, a therapeutic protein made by a method described herein, or a pharmaceutical composition described herein, for use in therapy.

In some embodiments, an auxotrophic engineered Plasmodium (or population of auxotrophic engineered Plasmodia) described herein (see, e.g., § 5.2.2.1) is administered to the subject. In some embodiments, a cognate compound of the auxotrophic engineered Plasmodium (or population of auxotrophic engineered Plasmodia) is co-administered to the subject. In some embodiments, if the subject exhibits an immune response or more than a mild immune response to the auxotrophic engineered Plasmodium (or population of auxotrophic engineered Plasmodia) the co-administration of the cognate compound is withheld or discontinued. In some embodiments, a IPP auxotrophic engineered Plasmodium (or population of IPP auxotrophic engineered Plasmodia) (e.g., as described herein) is administered to the subject, and IPP is co-administered to the subject. In some embodiments, if the subject exhibits an immune response or more than a mild immune response to the auxotrophic engineered Plasmodium (or population of auxotrophic engineered Plasmodia) the co-administration of the IPP is withheld or discontinued. Mild immune response includes, e.g., an immune response that does not necessitate discontinuation of the administered agent.

In some embodiments, the therapeutic protein made by a method described herein is administered to the subject, wherein the hRBCs utilized in the method of making the therapeutic protein, are autologous to the subject. In some embodiments, the therapeutic protein made by a method described herein is administered to the subject, wherein the hRBCs utilized in the method of making the therapeutic protein, are allogenic to the subject.

In one aspect, provided herein are method of treating, ameliorating, or preventing a disease in a subject in need thereof, the method comprising (a) obtaining a composition of hRBCs comprising an engineered Plasmodium (or population of engineered Plasmodia) described herein; (b) obtaining a sample of whole blood from the subject; (c) culturing the composition in the presence of the sample under conditions and for a period of time sufficient to allow for expression of the therapeutic protein; (d) isolating the serum from the hRBCs of the culture, wherein the serum comprises the expressed therapeutic protein; and (e) administering either (i) at least a portion of the serum comprising the therapeutic protein or (ii) the therapeutic protein, to the subject, to thereby treat the subject.

In some embodiments, the composition and the same are cultured under conditions and for a period of time sufficient for (i) release of the engineered Plasmodium from the hRBCs of the composition, (ii) infection of the population of hRBCs in the sample by the engineered Plasmodium, and (iii) expression of the therapeutic protein.

In some embodiments, the method further comprises isolating the therapeutic protein prior to administration to the subject. In some embodiments, the method further comprises purifying the therapeutic protein prior to administration to the subject.

In some embodiments, the therapeutic protein is secreted from the hRBCs. In some embodiments, the therapeutic protein is not secreted from the hRBCs.

In some embodiments, the culturing results in the rupture of hRBCs infected with the engineered Plasmodium. In some embodiments, the rupture results in the release of the therapeutic protein.

In some embodiments, the method further comprises measuring the concentration of therapeutic protein produced prior to administration to the subject. In some embodiments, the method further comprises measuring the concentration of therapeutic protein to determine the appropriate volume for administration to the subject.

In one aspect, provided herein are engineered Plasmodia described herein, therapeutic proteins made by a method described herein, or pharmaceutical compositions described herein for use a method of treating, ameliorating, or preventing a disease in a subject in need thereof, the method comprising administering to the subject the engineered Plasmodia described herein, the therapeutic protein made by a method described herein, or the pharmaceutical composition described herein. In some embodiments, the subject does not exhibit or only exhibits a mild symptomatic immune response to the engineered Plasmodium. In some embodiments, the therapeutic protein made by a method described herein is administered to the subject, wherein the hRBCs utilized in the method of making the therapeutic protein, are autologous to the subject. In some embodiments, the therapeutic protein made by a method described herein is administered to the subject, wherein the hRBCs utilized in the method of making the therapeutic protein, are allogenic to the subject.

In one aspect, provided herein are engineered Plasmodia described herein for use in a method of treating of a disease in a subject in need thereof, the method comprising (a) obtaining a composition of hRBCs comprising the engineered Plasmodium described herein; (b) obtaining a sample of whole blood from the subject; (c) culturing the composition in the presence of the sample under conditions and for a period of time sufficient to allow for expression of the therapeutic protein, (d) isolating the serum from the hRBCs of the culture, wherein the serum comprises the expressed therapeutic protein; and (e) administering either (i) at least a portion of the serum comprising the therapeutic protein or (ii) the therapeutic protein, to the subject.

In one aspect, provided herein is a use of an engineered Plasmodium described herein, a therapeutic protein made by a method described herein, or a pharmaceutical composition described herein for the manufacture of a medicament.

In one aspect, provided herein is a use of an engineered Plasmodium described herein, a therapeutic protein made by a method described herein, or a pharmaceutical composition described herein for the manufacture of a medicament. In a further aspect, provided herein is an engineered Plasmodium described herein, a therapeutic protein made by a method described herein, or a pharmaceutical composition described herein for use in therapy. In a further aspect, provided herein is an engineered Plasmodium described herein, a therapeutic protein made by a method described herein, or a pharmaceutical composition described herein for use in a method of treating, ameliorating, or preventing a disease in a subject in need thereof, the method comprising administering to the subject the engineered Plasmodia described herein, the therapeutic protein made by a method described herein, or the pharmaceutical composition described herein. In some embodiments, the subject does not exhibit or only exhibits a mild symptomatic immune response to the engineered Plasmodium. In some embodiments, the therapeutic protein made by a method described herein is administered to the subject, wherein the hRBCs utilized in the method of making the therapeutic protein, are autologous to the subject. In some embodiments, the therapeutic protein made by a method described herein is administered to the subject, wherein the hRBCs utilized in the method of making the therapeutic protein, are allogenic to the subject. Mild immune response includes, e.g., an immune response that does not necessitate discontinuation of the administered agent.

### 5.8 Kits

In one aspect, provided herein are kits comprising an engineered Plasmodium (or population of engineered Plasmodia) described herein, a population of RBCs comprising an engineered Plasmodium (or population of engineered Plasmodia) described herein, a therapeutic protein made by a method described herein (see, e.g., § 5.5), or a pharmaceutical composition described herein. In addition, the kit may comprise a liquid vehicle for solubilizing or diluting, and/or technical instructions. The technical instructions of the kit may contain information about administration and dosage and subject groups.

In some embodiments, the engineered Plasmodium (or population of engineered Plasmodia) described herein, the population of RBCs comprising an engineered Plasmodium (or population of engineered Plasmodia) described herein, the therapeutic protein made by a method described herein, or the pharmaceutical composition described herein is provided in a separate part of the kit, wherein the engineered Plasmodium (or population of engineered Plasmodia) described herein, the population of RBCs comprising an engineered Plasmodium (or population of engineered Plasmodia) described herein, the therapeutic protein made by a method described herein, or the pharmaceutical composition described herein is optionally lyophilized, spray-dried, or spray-freeze dried. The kit may further contain as a part a vehicle (e.g., buffer solution) for solubilizing the dried or lyophilized the engineered Plasmodium (or population of engineered Plasmodia) described herein, the population of RBCs comprising an engineered Plasmodium (or population of engineered Plasmodia) described herein, the therapeutic protein made by a method described herein, or the pharmaceutical composition described herein. In some embodiments, the kit comprises a single dose container. In some embodiments, the kit comprises a multi-dose container. In some embodiments, the kit comprises an administration device (e.g., an injector for intradermal injection or a syringe for intramuscular injection).

In some embodiments, the kit comprises a vessel (e.g., a syringe) comprising an engineered Plasmodium comprising in its genome a heterologous nucleic acid molecule encoding a therapeutic protein (e.g., an engineered Plasmodium described herein). In some embodiments, the kit comprises a vessel (e.g., a syringe) comprising an engineered Plasmodium described herein). In some embodiments, the kit comprises a vessel (e.g., a syringe) comprising an engineered Plasmodium comprising in its genome a heterologous nucleic acid molecule encoding a therapeutic protein (e.g., an engineered Plasmodium described herein) within a population of red blood cells (e.g., hRBCs). In some embodiments, the kit comprises a vessel (e.g., a syringe) comprising an engineered Plasmodium described herein) within a population of red blood cells (e.g., hRBCs). In some embodiments, the vessel comprises a population of RBCs (e.g., hRBCs) comprising an engineered Plasmodium comprising in its genome a heterologous nucleic acid molecule encoding a therapeutic protein (e.g., an engineered Plasmodium described herein). In some embodiments, the vessel comprises a population of RBCs comprising an engineered Plasmodium (or population of engineered Plasmodia) described herein.

In some embodiments, the vessel is a syringe, a tube, dish, or the like. In some embodiments, the vessel (e.g., a syringe) is configured for drawing blood from a subject (e.g., human subject). In some embodiments, the vessel is compatible with a device capable of obtaining a blood sample from a subject (e.g., a butterfly needle, syringe, etc.). In some embodiments, the vessel comprises a device capable of obtaining a blood sample from a subject (e.g., a butterfly needle, syringe, etc.). In some embodiments, the vessel is capable of supporting culture of cells (e.g., RBCs) (e.g., obtained from a subject). In some embodiments, the vessel is a cell culture vessel (e.g., syringe). In some embodiments, the vessel is capable of holding from about 1-100 mL, 1-90 mL, 1-80 mL, 1-70 mL, 1-60 mL, 1-50 mL, 1-40 mL, 1-30 mL, 1-20 mL, 1-10 mL, 1-5 mL, 5-50 mL, 5-40 mL, 5-30 mL, 5-20 mL, 5-10 mL of solution (e.g., whole blood). In some embodiments, the vessel is capable of holding at least about 1, 5, 10, 20, 30, 40, 50, 60, 70, 80, 90 or 100 mL of solution (e.g., whole blood). In some embodiments, the vessel is capable of holding about 1, 5, 10, 20, 30, 40, 50, 60, 70, 80, 90 or 100 mL of solution (e.g., whole blood).

In some embodiments, the vessel is configured such that serum and red blood cells within the vessel can be separated. In some embodiments, the vessel is configured such that serum and red blood cells within the vessel can be separated without centrifugation. In some embodiments, the vessel comprises a space for insertion (e.g., manual insertion) of a valve capable of separating serum and red blood cells within the vessel. In some embodiments, the vessel comprises a valve that can be manipulated to separate serum and red blood cells within the vessel.

In some embodiments, the kit comprises a temperature control device (e.g., a warmer). In some embodiments, the temperature control device (e.g., warmer) is compatible with the vessel such that when the temperature control device (e.g., warmer) is attached or otherwise associated with the vessel, the temperature control device (e.g., warmer) can control the temperature of the vessel (e.g., modulate (e.g., warm) the temperature of the contents of the vessel. In some embodiments, temperature control device (e.g., warmer) is capable of warming the contents of the vessel. In some embodiments, the temperature control device (e.g., warmer) is capable of warming the contents of the vessel (e.g., culture tube) to from about 30-45° C., 35-45° C., 37-45° C., 30-40° C., or 30-47° C. In some embodiments, temperature control device (e.g., warmer) is capable of warming the contents of the vessel. In some embodiments, the temperature control device (e.g., warmer) is capable of warming the contents of the vessel to at least about 30° C., 31° C., 32° C., 33° C., 34° C., 35° C., 36° C., 37° C., 38° C., 39° C., or 40° C. In some embodiments, the temperature control device (e.g., warmer) is capable of warming the contents of the vessel to about 30° C., 31° C., 32° C., 33° C., 34° C., 35° C., 36° C., 37° C., 38° C., 39° C., or 40° C. In some embodiments, the temperature control device (e.g., warmer) is capable of warming the contents of the vessel to about 37° C. In some embodiments, the temperature control device comprises a battery.

In some embodiments, the kit comprises a device capable of measuring the level, concentration, etc. of a protein. In some embodiments, the device is capable of measuring the concentration of a protein in the vessel. In some embodiments, the device is capable of measuring the concentration of a protein in serum. In some embodiments, the device is capable of measuring the concentration of a protein in serum in the vessel. In some embodiments, the device for measuring the level, concentration, etc. of a protein is a spectrophotometer. In some embodiments, the device for measuring the level, concentration, etc. of a protein is a spectrophotometer capable of measuring optical absorbance at 280 nm. In some embodiments, the device for measuring the level, concentration, etc. of a protein comprises a battery.

In some embodiments, the kit comprises an administration device capable of administering a therapeutic protein to a subject. In some embodiments, the kit comprises an administration device capable of injecting (e.g., intramuscular, subcutaneous, intradermal, intravenous) a therapeutic protein into a subject. In some embodiments, the kit comprises an administration device capable of infusing (e.g., intravenous infusion) a therapeutic a protein into a subject. In some embodiments, the administration device comprises a needle. In some embodiments, the administration device is compatible with the vessel. In some embodiments, the administration device is compatible with the vessel such that the device can be attached to the vessel for administration of the contents of the vessel (e.g., serum) to a subject.

In some embodiments, the kit comprises any one or more (e.g., 1, 2, or 3) of (i) a temperature control device configured to warm the vessel (e.g., optionally comprising a battery) (e.g., a temperature control device described herein); (ii) a device for measuring protein concentration (e.g., a spectrophotometer capable of measuring optical absorbance at 280 nm) (e.g., a device for measuring protein concentration described herein); and/or (iii) an administration device (e.g., an injection or infusion device) (e.g., a syringe) (e.g., configured to introduce a solution comprising a therapeutic protein into a subject, wherein the injection or infusion device is compatible with the vessel such that the device can be attached to the vessel for administration of the contents of the vessel into the subject) (e.g., an administration device described herein).

In some embodiments, the kit is designed for storage and/or transportation at from about 10-2° C. In some embodiments, the kit is designed for storage and/or transportation at from about 10-2° C., 10-4° C., 8-2° C., 8-4° C., 6-2° C., or 6-4° C. In some embodiments, the kit is designed for storage and/or transportation at about 10° C., 8° C., 6° C., 4° C., or 2° C. In some embodiments, the kit is designed for storage and/or transportation at about 4° C. In some embodiments, the kit is intended for storage and/or transportation at from about 10-2° C. In some embodiments, the kit is intended for storage and/or transportation at from about 10-2° C., 10-4° C., 8-2° C., 8-4° C., 6-2° C., or 6-4° C. In some embodiments, the kit is intended for storage and/or transportation at about 10° C., 8° C., 6° C., 4° C., or 2° C. In some embodiments, the kit is intended for storage and/or transportation at about 4° C.

Any of the kits described herein may be used in any of the methods described herein (see, e.g., § 5.8).

### 6. EXAMPLES

table OF CONTENTS
6.1. Example 1. Generation of Engineered Plasmodia
6.2 Example 2. Generation of Therapeutic Proteins in vitro using engineered Plasmodia
6.3 Example 3. Generation of Genetically Engineered Plasmodia for Protein Production
6.4 Example 4. Production of Therapeutic Proteins utilizing Genetically Engineered Plasmodia
6.5 Functional and Quantitative Characterization of Therapeutic Proteins

### 6.1 Example 1. Generation of Engineered Plasmodia

The following Example describes the introduction of a transgene encoding a therapeutic protein into Plasmodia, the selection of engineered Plasmodia, the assessment of transgene expression in the engineered Plasmodia, and the preservation of engineered Plasmodia.

Briefly, a DNA targeting vector comprising (i) a left homology arm targeting the Plasmodium HSP110 gene locus, (ii) a 2A skip peptide, (iii) a polynucleotide encoding the therapeutic protein of interest, such as plasma protease C1 inhibitor (C1-INH), (iv) an optional signal peptide comprising e.g., a PEXEL motif, and (v) a right homology arm is generated as described in Nasamu, A. S., Falla, A., Pasaje, C. F. A. et al. An integrated platform for genome engineering and gene expression perturbation in Plasmodium falciparum. Sci Rep 11, 342 (2021). https://doi.org/10.1038/s41598-020-77644-4 (hereinafter “Nasamu 2021”), the entire contents of which is incorporated by reference herein for all purposes. The polynucleotide encoding the therapeutic protein of interest is codon optimized for Plasmodia via reverse translation of the amino acid sequence of the protein of interest using the most abundant Plasmodium (e.g., NF54 P. falciparum) codons. The DNA targeting vector is introduced into the Plasmodia (e.g., NF54 P. falciparum) using standard methods known in the art and described herein (see, e.g., Ribeiro 2018), to thereby produce engineered Plasmodia.

The engineered Plasmodia (e.g., NF54 P. falciparum or a similar Plasmodium strain) are isolated through positive selection in blood culture, as described in Nasamu 2021. Transgene (a heterologous nucleic acid molecule) insertion is assessed via nucleotide sequencing of a PCR amplicon spanning the insertion site. Engineered Plasmodia populations are expanded in blood culture from a single transformed Plasmodium and cryopreserved as described in Stanisic, D. I., Liu, X. Q., De, S. L. et al. Development of cultured Plasmodium falciparum blood-stage malaria cell banks for early phase in vivo clinical trial assessment of anti-malaria drugs and vaccines. Malar J 14, 143 (2015). https://doi.org/10.1186/s12936-015-0663-x (hereinafter “Stanisic 2015”), the entire contents of which is incorporated by reference herein for all purposes. The therapeutic protein can be isolated from the supernatant of engineered Plasmodia/RBC cultures (for secreted proteins), or the lysates of engineered Plasmodia/RBC cultures (for non-secreted proteins) and its activity assessed using standard methods known in the art and described herein.

### 6.2 Example 2. Generation of Therapeutic Proteins In Vitro Using Engineered Plasmodia

The following Example describes the generation of secreted therapeutic proteins in vitro using engineered Plasmodia.

Briefly, cryopreserved ring-stage or merozoite stage genetically engineered Plasmodia (generated according to Example 1 § 6.1) expressing a therapeutic protein are used to initiate infection of in vitro RBC cultures established with blood sourced from an individual subject or from a blood bank, as described in Stanisic 2015. Following a period of time optimized for maximum expression and viability of the therapeutic protein, the culture media is collected and centrifuged to isolate the culture supernatant containing the secreted therapeutic protein from the Plasmodia infected RBCs. In some instances, the isolated supernatant is administered to a subject for therapeutic use. In some instances, the therapeutic protein is further isolated and/or purified using standard methods known in the art and described herein (e.g., affinity purification) prior to administration to a subject for therapeutic use.

### 6.3 Example 3. Generation of Genetically Engineered Plasmodia for Protein Production

The following example describes the generation of genetically engineered plasmodia comprising in its genome a heterologous nucleic acid encoding a therapeutic protein. The particular exemplary therapeutic proteins were chosen as model proteins for the invention in part for their significant complexity and tertiary and quaternary structure, such being multi-subunit proteins and/or having a high number of disulfide bonds.

Briefly, a nucleotide sequence encoding an anti-TNFα antibody (SEQ ID NOS: 1-2) and a nucleotide sequence encoding a dimeric fusion protein wherein each chain of the protein comprises the tumor necrosis factor receptor 2 (TNFR2) extracellular domain (LCD) operably connected to an Ig Fc region (SEQ ID NO: 3) were each cloned into the puc57-Hsp110 vector using SLIC (with MfeI and SpeI as described in (reference).

The amino acid sequence of the anti-TNF antibody and the dimeric TNFR2 ECD-Fc fusion protein are set forth in Table 3.

TABLE 3
The Amino Acid Sequence of Exemplary Therapeutic Proteins.

| | | | SEQ ID |
|---|---|---|---|
| Description | | Amino Acid Sequence | NO |
| Anti-TNFα | Heavy Chain | EVQLVESGGGLVQPGRSLRLSCAASGFTFDDYAMH | 1 |
| Antibody | | WVRQAPGKGLEWVSAITWNSGHIDYADSVEGRFTI | |
| | | SRDNAKNSLYLQMNSLRAEDTAVYYCAKVSYLSTA | |
| | | SSLDYWGQGTLVTVSSASTKGPSVFPLAPSSKSTS | |
| | | GGTAALGCLVKDYFPEPVTVSWNSGALTSGVHTFP | |
| | | AVLQSSGLYSLSSVVTVPSSSLGTQTYICNVNHKP | |
| | | SNTKVDKVEPKSCDKTHTCPPCPAPELLGGPSVFL | |
| | | FPPKPKDTLMISRTPEVTCVVVDVSHEDPEVKFNW | |
| | | YVDGVEVHNAKTKPREEQYNSTYRVVSVLIVLHQD | |
| | | WLNGKEYKCKVSNKALPAPIEKTISKAKGQPREPQ | |
| | | VYTLPPSRDELTKNQVSLTCLVKGFYPSDIAVEWE | |
| | | SNGQPENNYKTTPPVLDSDGSFFLYSKLIVDKSRW | |
| | | QQGNVFSCSVMHEALHNHYTQKSLSLSPGK | |
| | Light Chain | DIQMTQSPSSLSASVGDRVTITCRASQGIRNYLAW | 2 |
| | | YQQKPGKAPKLLIYAASTLQSGVPSRFSGSGSGTD | |
| | | FTLTISSLQPEDVATYYCQRYNRAPYTFGQGTKVE | |
| | | IKRTVAAPSVFIFPPSDEQLKSGTASVVCLLNNFY | |
| | | PREAKVQWKVDNALQSGNSQESVTEQDSKDSTYSL | |
| | | SSTLTLSKADYEKHKVYACEVTHQGLSSPVTKSFN | |
| | | RGEC | |

| | | |
|---|---|---|
| TNFR2 ECD-Fc Fusion | LPAQVAFTPYAPEPGSTCRLREYYDQTAQMCCSKC | 3 |
| Protein | SPGQHAKVFCTKTSDTVCDSCEDSTYTQLWNWVPE | |
| | CLSCGSRCSSDQVETQACTREQNRICTCRPGWYCA | |
| | LSKQEGCRLCAPLRKCRPGFGVARPGTETSDVVCK | |
| | PCAPGTFSNTTSSTDICRPHQICNVVAIPGNASMD | |
| | AVCTSTSPTRSMAPGAVHLPQPVSTRSQHTQPTPE | |
| | PSTAPSTSFLLPMGPSPPAEGSTGDEPKSCDKTHT | |
| | CPPCPAPELLGGPSVFLFPPKPKDTLMISRTPEVT | |
| | CVVVDVSHEDPEVKFNWYVDGVEVHNAKTKPREEQ | |
| | YNSTYRVVSVLIVLHQDWLNGKEYKCKVSNKALPA | |
| | PIEKTISKAKGQPREPQVYTLPPSREEMTKNQVSL | |
| | TCLVKGFYPSDIAVEWESNGQPENNYKTTPPVLDS | |
| | DGSFFLYSKLTVDKSRWQQGNVFSCSVMHEALHNH | |
| | YTQKSLSLSPGK | |

The repair plasmid contained homology sequences from the PfHsp110c gene (PF3D7_0708800). The repair sequences included the last 429 bp (not including the stop codon) from the PfHsp110c gene, followed by a 2A skip peptide, a modular tagging cassette, and the first 400 bps from PfHsp110c 3′UTR. The anti-TNFα antibody repair plasmid included the localization motif MSP1 (MKIIFFLCSFLFFIINTQCVTHE) (SEQ ID NO: 4), and a 2A skip peptide (GSGEGRGSLLTCGDVEENPGP) (SEQ ID NO: 5) between the heavy and light chain sequences. Two TNFR2 ECD—Fc fusion protein constructs were generated, one construct included the MSP1 localization motif while the other included the plasmodium export element (PEXEL) localization motif. All constructs were created using gene synthesis (GeneScript).

PCR products were inserted into the respective plasmids using sequence and ligation independent cloning as previously described by Florentin et al. (Florentin, A., Stephens, D. R., Brooks, C. F., Baptista, R. P., and Muralidharan, V. 2020. “Plastid biogenesis in malaria parasites requires the interactions and catalytic activity of the Clp proteolytic system.” Proc. Natl. Acad. Sci 13719-13729, the entire contents of which are incorporated herein by reference for all purposes (referred to herein as “Florentin 2020”))).

The repair plasmids prepared above along with a plasmid expressing a gRNA targeting the hsp110 locus were transfected into wild type P. falciparum 3D7 parasites. The 3D7 parasites were cultured at 37° C. in RPMI 1640 medium (with additional 25 mM HEPES, 15 mg/L hypoxanthine, 2.5 mg/L thymidine, 0.252% NaHCO₃, 0.2% glucose, 0.011% sodium pyruvate, 0.02% gentamicin and 0.25% AlbuMax I) under 5% O₂, 5% CO₂, 90% N₂ gas mix. The parasites were grown in 2% hRBCs. 150 μL of packed RBCs were transfected by electroporation with 25 μg total of endotoxin-free (Cas9+repair) plasmids and then infected at 0.5-1% parasitemia. Positive selection was initiated after 48 hours using 1 μM DSM1 for approximately 3 weeks and dropped when setting cloning plates.

Genomic integration of each of the two constructs, the PEXEL-TNFR2 ECD-Fc fusion protein and anti-TNFα antibody, was verified using the polymerase chain reaction (PCR) coupled with gel electrophoresis. As shown in FIG. 2, the PCR products corresponding to the anticipated sizes of the integrated constructs were observed—a distinct band at approximately 2.8 Kb for the PEXEL-TNFR2 ECD-Fc fusion protein (indicated with a red arrow) and another at around 3.4 Kb for the anti-TNFα antibody (indicated with blue arrows), thereby confirming accurate genomic incorporation of both constructs.

### 6.4 Example 4. Production of Therapeutic Proteins Utilizing Genetically Engineered Plasmodia

The following Example describes the generation of therapeutic proteins using the genetically engineered Plasmodia generated in Example 3.

Briefly, the supernatant of the cultures generated in Example 3 were utilized for western blot analysis. The western blots were performed as described previously by Florentin 2020 using commercially available versions of the anti-TNFα antibody (Invivosim Catalog No. SIM0001) and the TNFR2 ECD-Fc fusion protein (IchorBio LTD Catalog No. ICH4022) from as positive controls. Detection was done using human IgG Fc cross-adsorbed secondary antibody (SA5-10138). The Western blot images and quantifications were processed and analyzed using the Odyssey infrared imaging system software (LICOR Biosciences).

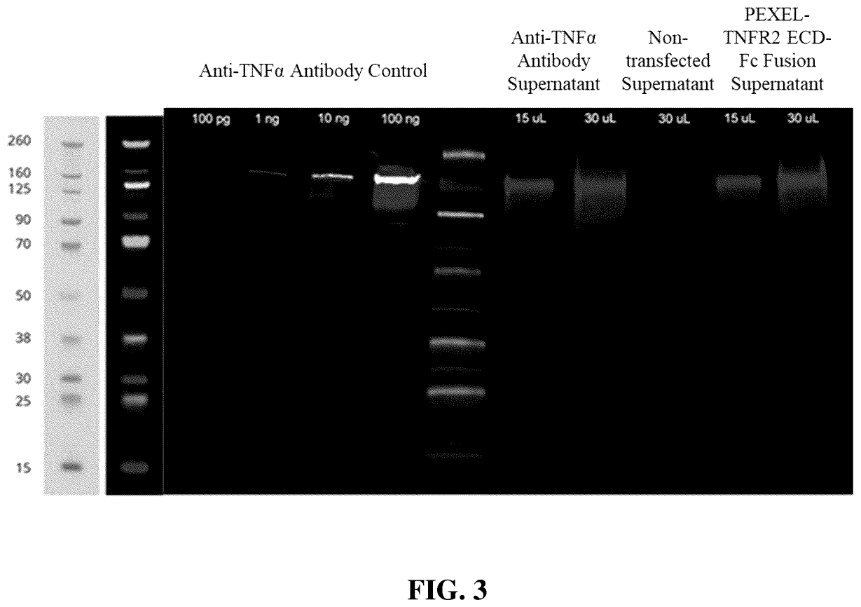

As shown in FIG. 3, both the anti-TNFα antibody (as evidenced by the protein band at 150 kDa) and the PEXEL-TNFR2 ECD-Fc fusion protein (as evidenced by the protein band at 148 KDa) were expressed by the genetically engineered plasmodia generated in Example 3.

### 6.5 Example 5. Functional and Quantitative Characterization of Therapeutic Proteins Produced Utilizing Genetically Engineered Plasmodia

The following example describes the functional and quantitative characterization of therapeutic proteins generated in Example 4.

An ELISA-based TNF-α binding assay was used to functionally and quantitatively evaluate the therapeutic proteins generated in Example 4. Briefly, 384-Maxisorp plates (Sigma, P6366-iCS) were coated with TNF-α (Acro Bio, TNA-H5228) at a concentration of 2 μg/mL in 1×ELISA coating buffer (Biolegend, 421701-BL). The plates were then stored overnight at 4° C. The following day, the plates were washed 3 times using 80 μL per well of 0.05% PBS-T (Fisher, PI28352) followed by a blocking step with 80 μL of Super Block buffer (Fisher, NC9782835) and a 1-hour incubation at room temperature on a 400 RPM shaker. Following a washing step, 25 μL of supernatant protein was added to each well as well as commercially available versions of the TNFR2 ECD-Fc fusion protein (Ichorbio LTD ICH4022) and anti-TNFα antibody (Invivosim, SIM000) (as controls) at half-log dose titration starting at 1 μg/mL, followed by a 1-hour room temperature incubation. After thoroughly washing the plate, 25 μL of goat anti-human IgG (H+L) secondary antibody HRP (Thermofisher, H10307), diluted at 1:2000 in Super Block, was added to each well and incubated for 1 hour at room temperature on the shaker and washed as previously described. Subsequently, 20 μL of TMB substrate (Thermofisher, 34028) was then added to each well, and incubated for 2 minutes in the dark at room temperature. Subsequently, 20 μL of Stop Solution (BioFX, LSTP-1000-01) was added to all wells, and the absorbance at 450 nm was measured using a Varioskan. Data analysis was performed using GraphPad Prism 10.2.

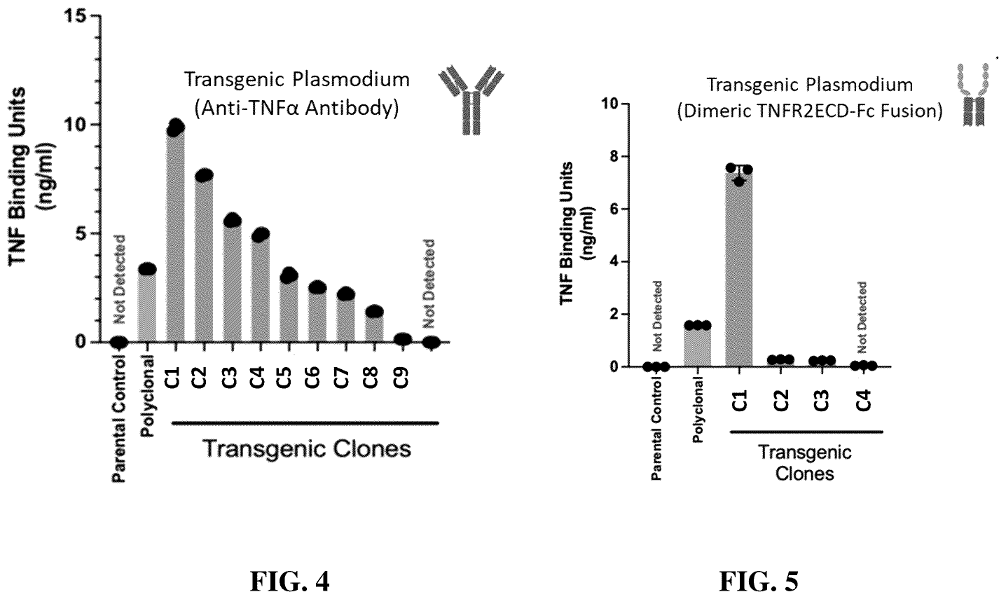

Evaluation of nine different transgenic clones (encoding the anti-TNFα antibody) showed that the majority of the clones (8 clones) generated detectable levels of anti-TNFα antibody capable of binding TNFα (FIG. 4). Clone C1 exhibited the highest production, reaching approximately −10 ng/ml. Clones C2, C3, and C4 also demonstrated anti-TNF binding activity, with levels ranging from approximately 8 ng/ml to just over 5 ng/ml (FIG. 4); clones 5, 6, 7, and 8 demonstrated anti-TNF binding activity, with levels ranging from approximately 1 ng/ml to 5 ng/ml (FIG. 4), and clone 8 showed lower but detectable levels of anti-TNF binding activity (FIG. 4). The parental control and polyclonal population served as baseline references, with the parental control showing no anti-TNF binding, whereas the polyclonal population displayed positive binding activity (FIG. 4).

Similarly, evaluation of four different transgenic clones (encoding the TNFR2 ECD-Fc fusion protein) showed the generation of detectable levels of TNFR2 ECD-Fc fusion protein capable of binding TNFα (TNFR2 ECD-Fc fusion protein) (FIG. 5). For example, clone C1 demonstrated expression of functional TNFR2 ECD-Fc fusion protein, with a TNF binding unit measurement of around 8 ng/ml. The parental control and polyclonal population served as baseline references, with the parental control showing no anti-TNF binding, whereas the polyclonal population displayed positive binding activity (FIG. 5).

These examples demonstrate that the methods, kits and compositions of the invention are able to produce functional therapeutic proteins, including highly complex multi-subunit proteins, and proteins with high numbers of disulfide bonds.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes. Other embodiments are within the following claims.

1. An engineered Plasmodium comprising a heterologous nucleic acid molecule within a genome of the Plasmodium, wherein the heterologous nucleic acid molecule encodes a therapeutic protein.

2.-22. (canceled)

23. A method of making an engineered Plasmodium, the method comprising:
(a) culturing a reference Plasmodium;
(b) genetically modifying the reference Plasmodium to introduce a heterologous nucleic acid molecule encoding a therapeutic protein into a genome of the reference Plasmodium;
to thereby make an engineered Plasmodium.

24.-50. (canceled)

51. A method of making a therapeutic protein, the method comprising:
culturing the engineered Plasmodium of claim 1 in a composition comprising hRBCs, under conditions and for a period of time sufficient for (i) infection of the hRBCs by the engineered Plasmodium and (ii) expression the therapeutic protein,
to thereby make a therapeutic protein.

52.-62. (canceled)

63. A method of making a therapeutic protein, the method comprising
culturing hRBCs comprising the engineered Plasmodium of claim 1 in the presence of a population of hRBCs under conditions and for a period of time sufficient for expression of the therapeutic protein,
to thereby make a therapeutic protein.

64.-75. (canceled)

76. A pharmaceutical composition comprising the engineered Plasmodium of claim 1, and a pharmaceutically acceptable excipient.

77. (canceled)

78. A kit comprising a vessel (i) configured for drawing blood from a human patient and (ii) comprising an engineered Plasmodium comprising in its genome a heterologous nucleic acid molecule encoding a therapeutic protein.

79. The kit of claim 78, further comprising any one or more of
(i) a temperature control device configured to warm the vessel;
(ii) a device for measuring protein concentration; and/or
(iii) an injection or infusion device configured to introduce a solution comprising a therapeutic protein into a subject, wherein the injection or infusion device is compatible with the vessel such that the device can be attached to the vessel for administration of the contents of the vessel into the subject.

80. (canceled)

81. The kit of claim 7, wherein the vessel comprises a population of hRBCs comprising the engineered Plasmodium.

82. (canceled)

83. A kit comprising a population of hRBCs comprising the engineered Plasmodium of claim 1.

84. (canceled)

85. The kit of claim 83, comprising a vessel comprising the population of hRBCs.

86.-87. (canceled)

88. The kit of claim 83, comprising a temperature control device.

89.-90. (canceled)

91. The kit of claim 83, comprising an administration device capable of administering a therapeutic protein into a subject.

92. (canceled)

93. A method of delivering a therapeutic protein to a subject, the method comprising: administering to the subject the therapeutic protein made by the method of claim 51, to thereby deliver the therapeutic protein to the subject.

94. A method of delivering an engineered Plasmodium a subject, the method comprising: administering to the subject the engineered Plasmodium of claim 1, to thereby deliver the engineered Plasmodium to the subject.

95. A method of treating, ameliorating, or preventing a disease in a subject in need thereof, the method comprising: administering to the subject the therapeutic protein made by the method of claim 51, to thereby treat, ameliorate, or prevent the disease in the subject.

96.-98. (canceled)

99. A method of treating, ameliorating, or preventing a disease in a subject in need thereof, the method comprising: administering to the subject the engineered Plasmodium of claim 1, to thereby treat, ameliorate, or prevent the disease in the subject.

100. (canceled)

101. A method of treating, ameliorating, or preventing a disease in a subject in need thereof, the method comprising
(a) obtaining a composition of hRBCs comprising the engineered Plasmodium of claim 1;
(b) obtaining a sample of whole blood from the subject;
(c) culturing the composition in the presence of the sample under conditions and for a period of time sufficient to allow for expression of the therapeutic protein;
(d) isolating the serum from the hRBCs of the culture, wherein the serum comprises the expressed therapeutic protein; and
(e) administering either (i) at least a portion of the serum comprising the therapeutic protein or (ii) the therapeutic protein, to the subject,
to thereby treat, ameliorate, or prevent the subject.

102.-119. (canceled)

120. A pharmaceutical composition comprising the engineered Plasmodium of claim 51, and a pharmaceutically acceptable excipient.

121. A kit comprising the therapeutic protein made by the method of 51.

122. A kit comprising the pharmaceutical composition of claim 76.


SEQUENCE LISTING (5 sequences)

SEQ ID NO: 1 - protein, 450 aa, Synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 467 aa, Synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 23 aa, Synthetic construct
MKIIFFLCSFLFFIINTQCVTHE

SEQ ID NO: 5 - protein, 21 aa, Synthetic construct
GSGEGRGSLLTCGDVEENPGP